(19) United States
(12) Patent Application Publication
(10) Pub. No.: US 2024/0352183 A1
(43) Pub. Date: Oct. 24, 2024

(54) CONDUCTIVE POLYMER WITH COVALENT CAPTURE ABILITY AND METHODS OF USING SAME

(71) Applicant: Duke University, Durham, NC (US)

(72) Inventors: Michael Tadross, Durham, NC (US); Isaac Weaver, Durham, NC (US)

(21) Appl. No.: 18/701,068

(22) PCT Filed: Oct. 13, 2022
(86) PCT No.: PCT/US2022/046618

§ 371 Date: Apr. 12, 2024

Related U.S. Application Data
(60) Provisional application No. 63/407,534, filed on Sep. 16, 2022.
(60) Provisional application No. 63/407,532, filed on Sep. 16, 2022.
(60) Provisional application No. 63/255,097, filed on Oct. 13, 2021.
(60) Provisional application No. 63/255,085, filed on Oct. 13, 2021.

(51) Int. Cl. C08G 61/12; C08F 112/14; H01B 1/12

(52) U.S. Cl. CPC C08G 61/126; C08F 112/30; H01B 1/127; C08G 2261/1424; C08G 2261/1432; C08G 2261/146; C08G 2261/3223; C08G 2261/94

(57) ABSTRACT

Disclosed herein are electrically conductive polymer compositions comprising a polymer and a dispersant, and methods of synthesizing the polymers therein. Also disclosed herein are microelectrode arrays comprising the electrically conductive material and a conductive polymer composition attached to a surface of the electrically conductive material. Further disclosed herein are methods of detecting electrical activity in a cell comprising contacting the microelectrode array with a cell and detecting electrical activity in the cell by covalently attaching the polymer of the conductive polymer composition to a protein on the surface of the cell.

### CROSS-REFERENCE TO RELATED APPLICATIONS

This claims priority to U.S. Provisional Patent Application No. 63/255,097, filed Oct. 13, 2021; U.S. Provisional Patent Application No. 63/407,532, filed Sep. 16, 2022; U.S. Provisional Patent Application No. 63/255,085, filed Oct. 13, 2021, and U.S. Provisional Patent Application No. 63/407,534, filed Sep. 16, 2022, each of which is incorporated herein by reference in its entirety.

### STATEMENT REGARDING FEDERALLY SPONSORED RESEARCH

This invention was made with government support under grant 1DP2MH1194025 awarded by the National Institutes of Health. The government has certain rights in the invention.

### FIELD

This disclosure relates to electrically conductive polymer compositions comprising a polymer and a dispersant, and methods of synthesizing the polymers therein. The disclosure also relates to microelectrode arrays comprising the electrically conductive material and a conductive polymer composition attached to a surface of the electrically conductive material. Further disclosed herein are methods of detecting electrical activity in a cell comprising contacting the microelectrode array with a cell and detecting electrical activity in the cell by covalently attaching the polymer of the conductive polymer composition to a protein on the surface of the cell.

### INTRODUCTION

Disfunction of neural circuits are thought to play a role in numerous brain disorders, including degenerative diseases such as Alzheimer's Disease and Parkinson's Disease, as well as psychiatric disorders like schizophrenia and depression. With the global neurological therapeutics market forecasted to reach almost $40 billion in 2024, it is imperative that the best tools are used to study and develop better therapeutics for neurological disorders. Understanding how neural circuits underlie brain function and encode behaviorally relevant information will require the ability to record, in freely behaving animals, from thousands of neurons simultaneously, with millisecond temporal precision, and knowledge of the genetic identity of each cell. Moreover, understanding how disease progression remodels neural ensembles will require recordings with months-long stability. To maximize impact, the underlying technology should be low-cost, scalable, and amenable to simple data storage and analysis. To date, no recording technology offers these features.

Extracellular electrodes can record thousands of cells with millisecond resolution, but provide little information about cell type, have limited stability owing to gliosis and drift, and suffer from a significant analysis bottleneck (i.e., the spike-sorting problem). Combining electrodes with “opto-tagging” offers some cell type-specificity (via expression of a light-sensitive opsin), but real-world performance is hampered by polysynaptic transmission, limiting yield to ˜10 neurons per animal. Conversely, genetically encoded indicators offer exquisite cell type-specificity, but have fundamental drawbacks: genetically encoded calcium indicators (GECIs) that enable stable recording from thousands of neurons of a genetically defined cell type are too slow to resolve millisecond action potentials—GECIs lack the temporal resolution to resolve single action potentials, and thus preclude examination of spike timing which is a key substrate in neural coding and Hebbian learning; faster (millisecond precision) genetically encoded voltage indicators (GEVIs) have limits on the number of GEVI molecules that can be expressed in a cell membrane and therefore GEVIs require two-photon optics that are incompatible with use in freely behaving animals, yield few cells in a field of view, and photo-bleach after ˜10 minutes of recording; and both GECIs and GEVIs require expensive volumetric optics, generate large datasets, and require sophisticated image registration and segmentation to extract underlying low-dimensional data.

Thus, there is a need for engineered conductive polymers and methods to covalently attach genetically defined neurons to electrodes to provide an interface to the genetically defined neurons in freely behaving animals that delivers cell type-specificity, millisecond temporal resolution, and months-long stability using inexpensive and scalable components.

### SUMMARY

In an aspect, the disclosure relates to a compound of formula (I)

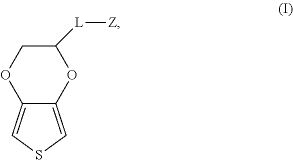

or a salt thereof, wherein: L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—; Z is a binding ligand selected from the group consisting of HaloTag, SNAP-tag, TMP-tag, βLac-tag, and CLIP-tag; and R^{a} is hydrogen or alkyl. In an embodiment, L is heteroalkylene-C(O)O-heteroalkylene-C(O)—, heteroalkylene-(O)NR^{a}-heteroalkylene-C(O)—, or heteroalkylene-OC(O)NR^{a}-heteroalkylene-C(O)—. In another embodiment, L is C₂₋₂₀heteroalkylene-C(O)O—C₂₋₆₀heteroalkylene-C(O)—, C₂₋₂₀heteroalkylene-(O)NR—C₂₋₆₀heteroalkylene-C(O)—, or C₂₋₂₀heteroalkylene-OC(O)NR^{a}—C₂₋₆₀heteroalkylene-C(O)—. In another embodiment, a compound as described herein is formula (I-a)

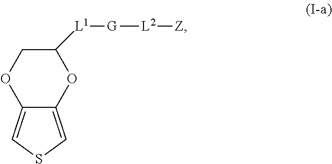

or a salt thereof, wherein: L¹ is C₂₋₁₀heteroalkylene; G is C(O)NR^{a} or C(O)O; L² is heteroalkylene-C(O)—; Z is a binding ligand selected from the group consisting of HaloTag, SNAP-tag, TMP-tag, βLac-tag, and CLIP-tag; and R^{a} is hydrogen or CO₁₋₃alkyl. In another embodiment, L¹ is C₂₋₆₀heteroalkylene; and L² is C₂₋₆₀heteroalkylene-C(O)—. In another embodiment, L¹ is C₂₋₆₀heteroalkylene, wherein 1 carbon of the heteroalkylene is replaced by O; and L² is C₂₋₆₀heteroalkylene-C(O)—, wherein at least 1 carbon of the heteroalkylene is replaced by O. In another embodiment, a compound as described herein is formula (I-b)

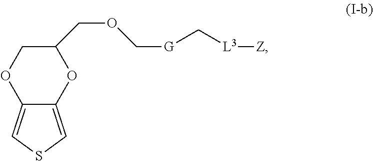

or a salt thereof, wherein: G is C(O)NR^{a} or C(O)O; L³ is (C—O—C)ₙ—, C—C(O)—; Z is a binding ligand selected from the group consisting of HaloTag, SNAP-tag, TMP-tag, βLac-tag, and CLIP-tag; R^{a} is hydrogen or CO₁₋₃ alkyl; and n is 1 to 40. In another embodiment, G is C(O)NH; L³ is (C—O—C)ₙ—C—C(O)—; Z is HaloTag, SNAP-tag, or CLIP-tag; and n is 1 to 10. In another embodiment, a compound as described herein is formula (I-c):

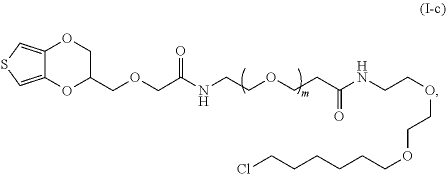

wherein: m is 1 to 40. In another embodiment, a compound as described herein is selected from the group consisting of:

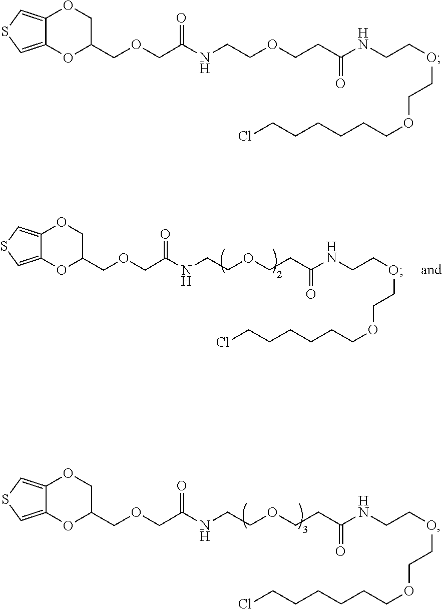

or a salt thereof.

In a further aspect, the disclosure relates to a conductive polymer composition comprising a polymer and a dispersant, the polymer including recurring units of formula (II)

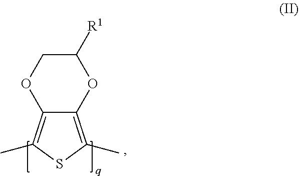

wherein: R¹ is hydrogen or

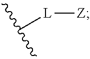

L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—; R^{a} is hydrogen or alkyl; Z is a binding ligand selected from the group consisting of HaloTag, SNAP-tag, TMP-tag, βLac-tag, and CLIP-tag; and q is 1 to 10,000,000. In an embodiment, the dispersant comprises polystyrene sulfonate (PSS), CIO₄, BF₄⁻, PF₆⁻, bis(trifluoromethylsulfonyl)imide (BTFMSI), C₆H₅O₇⁻³, CO₃⁻², S₂O₃⁻², C₂H₃O₂⁻, HPO₄⁻, H₂PO₄⁻, Cl⁻ Br⁻, NO₃⁻, or a combination thereof. In another embodiment, the polymer is a copolymer comprising recurring unit of formula (II-a)

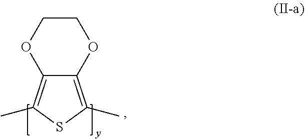

wherein y is 1 to 10,000,000; and recurring units of formula (II-b)

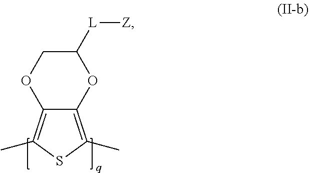

wherein z is 1 to 10,000,000. In another embodiment, L is C₂₋₂₀heteroalkylene-C(O)O—C₂₋₆₀heteroalkylene-C(O)—, C₂₋₂₀heteroalkylene-C(O)NR^{a}—C₂₋₂₀heteroalkylene-C(O)—, or C₂₋₂₀heteroalkylene-OC(O)NR^{a}—C₂₋₂₀heteroalkylene-C(O)—; and Z is HaloTag, SNAP-tag, or CLIP-tag. In another embodiment, the composition includes the polymer and the dispersant at a concentration ratio of about 0.001:1 to about 10:1 (polymer:dispersant). In another embodiment, the dispersant comprises PSS, ClO₄⁻, BF₄⁻, or a combination thereof. In another embodiment, the copolymer is a random copolymer, an alternating copolymer, or a block copolymer.

Another aspect of the disclosure provides a method of electrochemically synthesizing a conductive polymer, the method comprising adding a first mixture to an electrically conductive material, the first mixture comprising a solvent, a polystyrene sulfonate (PSS), and a 3,4-ethylenedioxythiophene (EDOT); applying an electrical current to the electrically conductive material to provide a poly(3,4-ethylenedioxythiophene) (PEDOT) on a surface of the electrically conductive material; adding a second mixture to the electrically conductive material, the second mixture comprising a solvent, a dispersant having a molecular weight of less than 1 kDa, and a compound of formula (I)

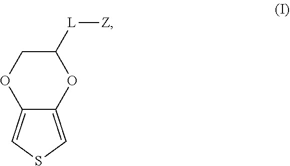

or a salt thereof, wherein: L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—; Z is a binding ligand selected from the group consisting of HaloTag, SNAP-tag, TMP-tag, βLac-tag, and CLIP-tag; and R^{a} is hydrogen or alkyl; and applying an electrical current to the electrically conductive material to provide a second polymer attached to the PEDOT, the second polymer including recurring units of formula (II-b)

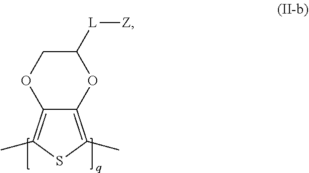

wherein: L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—; Z is a binding ligand selected from the group consisting of HaloTag, SNAP-tag, TMP-tag, βLac-tag, and CLIP-tag; R^{a} is hydrogen or alkyl; and z is 1 to 10,000,000. In an embodiment, the dispersant of the second mixture comprises BF₄⁻, PF₆⁻, BTFMSI, C₆H₅O₇⁻³, CO₃⁻², S₂O₃⁻², C₂H₃O₂⁻, HPO₄⁻², H₂PO₄⁻, Cl⁻, Br⁻, NO₃⁻, or a combination thereof. In another embodiment, L is C₂₋₂₀heteroalkylene-C(O)O—C₂₋₆₀heteroalkylene-C(O)—, C₂₋₂₀heteroalkylene-C(O)NR^{a}—C₂₋₆₀heteroalkylene-C(O)—, or C₂₋₂₀heteroalkylene-OC(O)NR^{a}—C₂₋₆₀heteroalkylene-C(O)—; and Z is HaloTag, SNAP-tag, or CLIP-tag.

Another aspect of the disclosure provides a microelectrode array comprising: an electrically conductive material; and a conductive polymer composition as described herein attached to a surface of the electrically conductive material. In an embodiment, the electrically conductive material is one or more of gold, platinized-platinum, tungsten, platinum, platinum iridium, tantalum pentoxide, titanium nitride, and indium tin oxide.

Another aspect of the disclosure provides a method of detecting electrical activity in a cell, the method comprising: contacting a microelectrode array as described herein with a cell; and detecting electrical activity in the cell by covalently attaching the polymer of the conductive polymer composition to a protein on the surface of the cell. In an embodiment, the microelectrode array is implanted into a subject. In another embodiment, the cell is capable of depolarizing. In another embodiment, the cell is one or more of neurons, muscle cells, endocrine cells, keratinocytes, glia, and cell lines expressing voltage-gated ion channels. In another embodiment, the protein on the surface of the cell is a genetically encoded protein. In another embodiment, the genetically encoded protein is delivered to the cell with a viruS. In another embodiment, detecting the electrical activity comprises measuring a change in voltage, a change in current, or a combination thereof. In another embodiment, the change in voltage, the change in current, or a combination thereof is used for decoding neural connectivity, frequency of firing, or a combination thereof. In another embodiment, the protein on the surface of the cell is one or more of HaloTag protein, SNAP-tag protein, TMP-tag protein, βLac-tag protein, and CLIP-tag protein.

The disclosure provides for other aspects and embodiments that will be apparent in light of the following detailed description and accompanying figures.

### BRIEF DESCRIPTION OF THE DRAWINGS

FIG. 1 is a diagram showing the chemical structure of an EDOT monomer and polymerization of the monomer into PEDOT:PSS. The chemical structure of PEDOT:PSS polymer chain and the location of the EDOT monomer in the PEDOT:PSS polymer are also shown. “n” as represented in this figure does not correlate to n as shown in formula (I), but rather are merely illustrative of the polymers having multiple repeating units.

FIG. 2A is a graph showing the mass spectra reading of EDOT-PEG3-HTL.1 and the insert shows the generic chemical structure of an EDOT-PEGₙ-HTL monomer where n=1, 2, 3, etc. “n” as represented in this figure does not correlate to n as shown in formula (I), but rather are merely illustrative of the polymers having multiple repeating units. FIG. 2B is a graph showing the X-ray photoelectron spectroscopy (XPS) spectra reading of polymerized EDOT-PEG3-HTL.1 and FIG. 2C is an XPS spectra reading of EDOT in the two layer configuration. The CL2p peak is towards the right-hand side of the graph. There is a slightly elevated peak for the EDOT-PEG3-HTL.1 polymerized plot.

FIG. 3A is an image showing polymerization of PEDOT:PSS on a gold surface. FIG. 3B is an image showing polymerization of PEDOT^{PEG[n]HTL}:PSS on a gold surface.

FIGS. 4A-C are diagrams showing polymerization of novel EDOT-R monomers into polymers. “n” as represented in these figures does not correlate to n as shown in formula (I), but rather are merely illustrative of the polymers having multiple repeating units. FIG. 4A is a diagram showing a PEDOT-R polymer with a PSS counter ion. FIG. 4B is a diagram showing a PEDOT-R polymer with lithium tetrafluoroborate (LiBF₄) counter ions. FIG. 4C is a diagram showing a PEDOT-R polymer with lithium perchlorate. Note: PEDOT-R denotes PEDOT polymer made up of novel EDOT-R monomer with R chain shown in FIG. 2A.

FIGS. 5A-F are diagrams showing graphical representations of single stage polymerization and co-polymerization of polymers shown in FIGS. 4A-C of novel EDOT-R monomers into polymers on a conductive material. FIG. 5A is a diagram of a PEDOT-R polymer with a PSS counter ion. FIG. 5B is a diagram of a PEDOT-R polymer with lithium tetrafluoroborate (LiBF₄) counter ions. FIG. 5C is a diagram of a PEDOT-R polymer with lithium perchlorate (LiClO₄) counter ions. FIG. 5D is a diagram of a PEDOT^{PEG3-HTL} polymer with PSS counter ions co-polymerized with PEDOT:PSS. FIG. 5E is a diagram of a PEDOT^{PEG3-HTL} polymer with lithium tetrafluoroborate (LiBF₄) counter ions co-polymerized with PEDOT:PSS. FIG. 5F is a diagram of a PEDOT^{PEG3-HTL} polymer with lithium perchlorate counter ions co-polymerized with PEDOT:PSS. Note: PEDOT-R denotes PEDOT polymer made up of novel EDOT-R monomer with R chain shown in FIG. 2A.

FIGS. 6A-C are diagrams showing two step polymerization of EDOT^{PEG3-HTL} monomers and PEDOT:PSS. FIG. GA is a diagram of PEDOT:PSS polymerization followed by PEDOT^{PEG3-HTL} polymerization with a PSS counter ion. FIG. 6B is a diagram of PEDOT:PSS polymerization followed by PEDOT^{PEG3-HTL} with lithium perchlorate counter ions. FIG. 6C is a diagram of PEDOT:PSS polymerization followed by PEDOT^{PEG3-HTL} with lithium tetrafluoroborate (LiBF₄) counter ions.

FIG. 7 is a diagram showing the most successful polymerization of the EDOT-monomers using a two-step polymerization method, showing the base PEDOT:PSS polymerization followed by the polymerization of PEDOT^{PEG3-HTL1} into a polymer with LiClO₄ counter-ions used for the second stage of polymerization and the location of the PEDOT^{PEG3-HTL1} in the polymer, “n” as represented in this figure does not correlate to n as shown in formula (I), but rather are merely illustrative of the polymers having multiple repeating units.

FIG. 8 is a pair of images showing a gold strip being polymerized with two layer PEDOT:PSS/PEDOT^{HTL}:LiClO₄.

FIG. 9A is a diagram showing the steps in a multi-step procedure for polymerization on conductive material of a polymer developed including PEDOT:PSS and incorporation of PEDOT^{PEG3-HTL} structure where PEDOT^{PEG3-HTL} coated gold strips were incubated with HTP^{βLac}, washed, and assayed for CCF2 breakdown. The bottom row shows a negative control for nonspecific adhesion where HTP^{βLac} pre-blocked with dummy HTL (gray triangle). FIG. 9B is a graph showing plots of the CCF2 conversion rate for the HTP^{βLac}. FIG. 9C is a series of graphs showing the CCF2 conversion rate for the negative control. The graphs for Condition 1.0 and Condition 2.0 show little to no nonspecific adhesion for the negative control. Shading is mean±SEM of 12 samples. FIG. 9D is a series of graphs showing a comparison of the polymer in ITO/glass (right), gold on polyamide plastic (left), and gold on silicon (middle), using the Beta Lactamase Assay. The top row shows an experimental case with HTL bound to HTP^{βLac}. The bottom row shows an appropriate control for each column. The polymerization protocol (using the PEDOT:PSS base+ PEDOT-PEG3-HTL:LiClO₄) is robust to different conductive substrates.

FIG. 9E is an image showing an example polymerization assay on gold-coated polyamide (as an example conductor) with multiple samples polymerized with different conditions to develop the polymer composition described herein. FIG. 9F are two images showing a proof of polymerization using a method as described herein on an ITO coated glass sample. FIG. 9G is an image showing an example binding assay in progress on gold/polyimide samples in which polymerized samples are being washed of non-specifically bound HTP^{βLac} proteins for evaluation in the HTP^{βLac} binding assay. This is one of the assays in which multiple samples polymerized with different conditions were tested to develop the polymer composition described herein. FIG. 9H is an image showing an example binding assay in progress on gold coated insulated silicon die and ITO coated glass die samples in which polymerized samples are being washed of non-specifically bound HTP^{βLac} proteins for evaluation in the HTP^{βLac} binding assay. This is one of the assays in which multiple samples polymerized with different conditions were tested to develop the polymer composition as described herein.

FIG. 10 is a pair of scanning electron microscope (SEM) images of an indium tin oxide (ITO) electrode pad before (left) and after (right) electrodeposition of platinized platinum to increase surface topography.

FIG. 11 is a series of images showing HTP− and HTP+ neuronal coverslips following identical addition and washoff of HTL-beads; retention was visually apparent on only the HTP+ coverslip. An image of confocal 3D reconstruction of HTP+ neuronal morphology is shown on the right via cytosolic florescent protein. White arrows indicate location of the HTL beads, inferred from spherical indentations.

FIG. 12A is a series of images showing a microelectrode array (MEA) made of glass and ITO with zoomed images of the polymerized electrode array. FIG. 12B is a series of images showing an MEA made of silicon and gold with zoomed images of the polymerized electrode array. FIG. 12C is a diagram showing a step-by-step procedure for patterning ITO and SU-8 layers, including photolithographic steps. FIG. 12D is a schematic showing the recording interface used to collect cell culture data, showing a custom circuit board and an exploded view of the full recording clam-shell. FIG. 12E is a diagram of an HTP− configuration featuring a double-dead HaloTag protein (ddHTP). FIG. 12F is an example recording from the HTP− device where the black line shows the average over recorded spikes with each individual spike in grey (left) and an image of the MEA pad (indicated with white x) corresponding to the recording shown (right). A DIC contrast image is overlaid with ΔF of GCaMP signal (green).

FIG. 12G is a diagram of an HTP+ configuration identical for FIG. 12C except for the potential of covalent bond formation. FIG. 12H is an example recording from the HTP+ device where the red line shows the average over recorded spikes with each individual spike in grey (left) and an image of the MEA pad (indicated with white x) corresponding to the recording shown (right). A DIC contrast image is overlaid with ΔF of GCaMP signal (green). FIG. 12I is a histogram of all detected spikes, binned by spike amplitude. Black is the composite of 225 pads from 6 HTP-devices and grey is the composite of 216 pads from 5 HTP+ devices.

FIG. 13A is a diagram showing an in vivo PEDOT-HTL.1 coated Michigan style penetrating microelectrode array (insert) and implantation of a packaged penetrating microelectrode array in a mouse using a PCB connector. FIG. 13B is a diagram showing *HTP and ^{dd}HTP conditions. FIG. 13C is an image showing an implanted mouse with an attached headstage during a recording session. FIG. 13D is an image showing a 32-channel in vivo probe.

### DETAILED DESCRIPTION

Described herein is genetically encoded electrophysiology that comprises expressing genetically encoded proteins on the surface of cells capable of depolarizing, such as neurons, to covalently attach the proteins to custom electrodes. The disclosure overcomes prior limitations: because cell type-specificity does not involve an optical element, there is no need for imaging or image analysis; because recordings are electrical, they provide millisecond temporal precision, ease of data acquisition, and potential scalability; with regard to stability, covalent attachment minimizes drift, and gliosis would not attenuate signals if glial cells do not invade the interface between electrode and neuron; and, by attaching one cell per electrode, spike sorting is amenable to automated algorithms, as electrical signal from a conjugated cell would be amplified (due to proximity) and signal from other cells attenuated (due to shielding by the attached cell) thereby improving signal to noise ratio.

In an embodiment, novel chemical derivatives of 3,4-ethylenedioxythiophene monomers were synthesized to contain a HaloTag Ligand (PEDOT-HTL). The HaloTag Protein (HTP) was transfected into a neuronal population, causing an HTP ligand to be expressed on the surface of the neurons. This facilitated covalent binding of the specific neuronal population expressing HTP to the PEDOT-HTL electrodes. This may be used in vitro or in vivo to preform electrophysiological recordings in freely moving subjects.

Therefore, the disclosure allows for stable neural activity recording in specific cell populations, is capable of recording with millisecond precision, in vitro or in vivo, does not require complex analysis procedures, and is cost-effective, scalable and modular.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 66, 6.7, 6.8, 6.9, and 7.0 are explicitly contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

The term “alkyl,” as used herein, refers to a straight or branched, saturated hydrocarbon chain containing from 1 to 20 carbon atoms. The term “lower alkyl” or “C₁-C₆ alkyl” means a straight or branched chain hydrocarbon containing from 1 to 6 carbon atoms. The term “C₁-C₄ alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and n-dodecyl.

The term “alkylene,” as used herein, refers to a divalent group derived from a straight or branched chain hydrocarbon of 1 to 100 carbon atoms, for example, of 2 to 60 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. A control may be a subject or cell without a monomer or polymer as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

The term “dispersant” as used herein, refers to an anionic species, such as an ionomer or counter ion. The ionomer (e.g., PSS) and/or counter ion (e.g., BF₄⁻) can have an anionic charge. The ionomer and/or counter ion can also include ionizable groups, or ionized groups. As used herein the term “ionizable groups,” means potentially ionic groups. The ionizable groups are such that they are readily converted to their anionic form during the dispersion/polymer preparation method as discussed below. Specific examples of anionic groups include carboxylate and sulfonate groups. The dispersant can exhibit an ionic interaction with the PEDOT and variants thereof. Accordingly, a dispersant can be introduced into a conductive polymer composition and can produce a “macromolecular salt” due to its anionic characteristics. This macromolecular salt can aid in providing the conductive polymer.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. For example, a genetic construct may encode a HaloTag protein that may be expressed in a cell of interest.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

The term “heteroalkylene” as used herein, means an alkylene group, as defined herein, in which one or more of the carbon atoms has been replaced by a heteroatom selected from S, Si, O, P and N. An example heteroalkylene includes, but is not limited to, a polyether, such as polyethylene glycol (PEG).

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins and receptors. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

“Vector” as used herein means a nucleic acid sequence containing an origin of replication. A vector may be a viral vector, bacteriophage, bacterial artificial chromosome, or yeast artificial chromosome. A vector may be a DNA or RNA vector. A vector may be a self-replicating extrachromosomal vector, and preferably, is a DNA plasmid. For example, the vector may encode a protein that binds a ligand as described herein such as a HaloTag protein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Monomers

Provided herein are compounds (also referred to as monomers) that can be used to provide polymers that can be conductive and that can interact (e.g., covalently) with proteins attached to a cell's surface. The compound can be of formula (I)

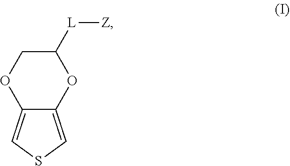

or a salt thereof, wherein: L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}— heteroalkylene-C(O)—, or heteroalkylene-OC(O)NR^{a}-heteroalkylene-C(O)—; Z is a binding ligand selected from the group consisting of HaloTag, SNAP-tag, TMP-tag, βLac-tag, and CLIP-tag; and R^{a} is hydrogen or alkyl. Salts of the compounds can also be pharmaceutically acceptable salts thereof.

L is a linker attaching Z (e.g., a binding ligand) to the 3,4-ethylenedioxythiophene core structure. Any suitable linker or linking mechanism known within the art can be used to attach Z to the core structure. For example, suitable linkers include, but are not limited to peptides, nucleic acids, polymers, ester linkages, amide linkages, carbamate linkages, PEG linkers, carbon chains, and the like. The linker or any part of the linker may be optionally substituted. In some embodiments, the linker is optionally substituted with halogen, ═O (oxo), ═S (thioxo), cyano, nitro, alkyl, alkenyl, alkynyl, haloalkyl, heteroalkyl, hydroxy, hydroxyalkyl, alkoxy, alkoxyalkyl, amino, alkylamino, acylamino, aminoalkyl, arylamino, —COOH, ketone, amide, carbamate, acyl, or a combination thereof. In addition, the linker can include a heteroalkylene that is optionally substituted.

In some embodiments, L is heteroalkylene-C(O)O-heteroalkylene-C(O)—, heteroalkylene-C(O)NR^{a}-heteroalkylene-C(O)—, or heteroalkylene-OC(O)NR-heteroalkylene-C(O)—. In some embodiments, L is heteroalkylene-C(O)O-heteroalkylene-C(O)— or heteroalkylene-C(O)NR^{a}-heteroalkylene-C(O)—. In some embodiments, L is heteroalkylene-C(O)NR^{a}-heteroalkylene-C(O)—.

In some embodiments, L is C₂₋₂₀heteroalkylene-C(O)O—C₂₋₆₀heteroalkylene-C(O)—, C₂₋₂₀heteroalkylene-C(O)NR^{a}—C₂₋₆₀heteroalkylene-C(O)—, or C₂₋₂₀heteroalkylene-OC(O)NR^{a}—C₂₋₆₀heteroalkylene-C(O)—. In some embodiments, L is C₂₋₂₀heteroalkylene-C(O)O—C₂₋₆₀heteroalkylene-C(O)— or C₂₋₂₀heteroalkylene-C(O)NR^{a}—C₂₋₆₀heteroalkylene-C(O)—. In some embodiments, L is C₂₋₂₀heteroalkylene-C(O)NR^{a}—C₂₋₆₀heteroalkylene-C(O)—.

In some embodiments, L includes PEG (e.g., (C—O—C)ₙ). For example, L can be heteroalkylene-C(O)O—Y¹—C(O)— or heteroalkylene-C(O)NR^{a}—Y¹—C(O)—, wherein Y¹ is —C—(C—O—C)ₙ—C—; and n is 1 to 40. In some embodiments, L is heteroalkylene-C(O)O—Y¹—C(O)— or heteroalkylene-C(O)NR^{a}—Y¹C(O)—, wherein Y¹ is —C—(C—O—C)ₙ—C—; and n is 1 to 20. In some embodiments, L is heteroalkylene-C(O)O—Y¹—C(O)—or heteroalkylene-C(O)NR^{a}—Y¹—C(O)—, wherein Y¹ is —C—(C—O—C)ₙ—C—; and n is 1 to 10.

Z is a binding ligand that can attach the monomer and polymer thereof to a specific protein expressed on a surface of a cell. In some embodiments, the binding ligand can allow for covalent attachment to a protein expressed on a surface of a cell. Z can be attached to L via methods known within the art. For example, Z and L may each individually have functional groups that are complimentary to each other in that they can form a covalent bond between the functional groups under appropriate conditions. Representative complimentary functional groups that can form a covalent bond include, but are not limited to, an amine and an activated ester, an amine and an isocyanate, an amine and an isothiocyanate, an amine and a carbonate, thiols for formation of disulfides, an aldehyde and amine for enamine formation, and an azide for formation of an amide via a Staudinger ligation. Depending on the functional groups, different bonds or linkages can be formed between Z and L. In some embodiments, Z has an amine at one end that can interact with, e.g., a carboxylic acid of L to form an amide bond. In some embodiments, Z is attached to L as

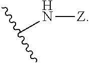

Z can be a number of different binding ligands that can interact with a protein on the surface of a cell. Example binding ligands include, but are not limited to, HaloTag, SNAP-tag, TMP-tag, βLac-tag, and CLIP-tag. These example binding ligands can be purchased from various biotechnology companies. For example, HaloTag can be purchased form Promega (see, e.g., Methods Mol Biol. 2007; 356:195-208, which is incorporated by reference herein in its entirety); SNAP-tag and CLIP-tag can be purchased from New England Biolabs (see, e.g., Nat Biotechnol. 2003 January; 21(1):86-9, which is incorporated by reference herein in its entirety); and TMP-tag can be purchased from Active Motif (see, e.g., ACS Chem Biol. 2009 Jul. 17; 4(7):547-56, which is incorporated by reference herein in its entirety).

In some embodiments, Z is HaloTag, SNAP-tag, TMP-tag, or CLIP-tag. In some embodiments, Z is HaloTag, SNAP-tag, or CLIP-tag. In some embodiments, Z is HaloTag. For example, Z can be:

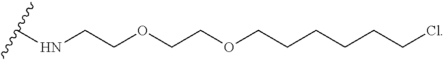 [SMILES: *NCCOCCOCCCCCCCl]

In some embodiments, L is C₂₋₂₀heteroalkylene-C(O)O—C₂₋₂₀heteroalkylene-C(O)—, C₂₋₂₀heteroalkylene-C(O)NR^{a}—C₂₋₆₀heteroalkylene-C(O)—, or C₂₋₂₀heteroalkylene-OC(O)NR^{a}—C₂₋₆₀heteroalkylene-C(O)—; and Z is HaloTag, SNAP-tag, or CLIP-tag. In some embodiments, L is C₂₋₁₀heteroalkylene-C(O)O—C₂₋₄₀heteroalkylene-C(O)—, C₂₋₂₀heteroalkylene-C(O)NR^{a}—C₂₋₄₀ heteroalkylene-C(O)—, or C₂₋₁₀heteroalkylene-OC(O)NR^{a}—C₂₋₄₀heteroalkylene-C(O)—; and Z is HaloTag, SNAP-tag, or CLIP-tag. In some embodiments, L is C₂₋₁₀heteroalkylene-C(O)O—C₂₋₄₀heteroalkylene-C(O)—, C₂₋₁₀ heteroalkylene-C(O)NR^{a}—C₂₋₄₀heteroalkylene-C(O)—, or C₂₋₁₀heteroalkylene-OC(O)NR^{a}—C₂₋₄₀heteroalkylene-C(O)—; and Z is HaloTag.

In some embodiments, the compound has formula (I-a)

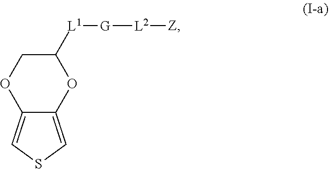

or a salt thereof, wherein: L¹ is C₂₋₁₀heteroalkylene; G is C(O)NR^{a} or C(O)O:L² is heteroalkylene-C(O)—; Z is a binding ligand selected from the group consisting of HaloTag, SNAP-tag, TMP-tag, βLac-tag, and CLIP-tag; and R^{a} is hydrogen or C₁₋₃alkyl.

In some embodiments, L¹ is C₂₋₆heteroalkylene; and L² is C₂₋₆₀heteroalkylene-C(O)—. In some embodiments, L¹ is C₂₋₆₀heteroalkylene; and L² is C₂₋₆₀heteroalkylene-C(O)—.

In some embodiments, L¹ is C₂₋₆heteroalkylene, wherein 1 carbon of the heteroalkylene is replaced by 0 or N; and L² is C₂₋₆₀heteroalkylene-C(O)—, wherein at least 1 carbon of the heteroalkylene is replaced by O or N, In some embodiments, L¹ is C₂₋₆heteroalkylene, wherein 1 carbon of the heteroalkylene is replaced by 0; and L² is C₂₋₆heteroalkylene-C(O)—, wherein at least 1 carbon of the heteroalkylene is replaced by O.

In some embodiments, the compound has formula (I-b)

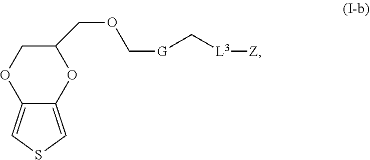

or a salt thereof, wherein: G is C(O)NR^{a} or C(O)O; L³ is (C—O—C)ₙ—C—C(O)—; Z is a binding ligand selected from the group consisting of HaloTag, SNAP-tag, TMP-tag, βLac-tag, and CLIP-tag; R^{a} is hydrogen or C₁₋₃alkyl; and n is 1 to 40.

In some embodiments, Z is HaloTag, SNAP-tag, or CLIP-tag. In some embodiments, Z is HaloTag.

In some embodiments, n is 1 to 30, 1 to 20, 1 to 15, 1 to 10, or 1 to 5. In some embodiments, n is greater than 1, greater than 2, greater than 3, greater than 4, greater than 5, greater than 10, greater than 15, greater than 20, or greater than 30. In some embodiments, n is less than 40, less than 35, less than 30, less than 25, or less than 20.

In some embodiments, G is C(O)NH; L³ is (C—O—C)ₙ—C—C(O)—; Z is HaloTag, SNAP-tag, or CLIP-tag; and n is 1 to 20. In some embodiments, G is C(O)NH; L³ is (C—O—C)ₙ—C—C(O)—; Z is HaloTag, SNAP-tag, or CLIP-tag; and n is 1 to 15. In some embodiments, S is C(O)NH; L³ is (C—O—C)ₙ—C—C(O)—; Z is HaloTag, SNAP-tag, or CLIP-tag; and n is 1 to 10. In some embodiments, S is C(O)NH; L³ is (C—O—C)˜—C—C(O)—; Z is HaloTag; and n is 1 to 10.

In some embodiments, the compound has formula (I-c)

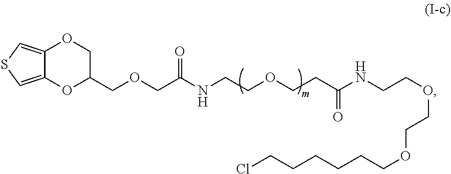

wherein: m is 1 to 40.

In some embodiments, m is 1 to 30, 1 to 20, 1 to 15, 1 to 10, or 1 to 5. In some embodiments, m is greater than 1, greater than 2, greater than 3, greater than 4, greater than 5, greater than 10, greater than 15, greater than 20, or greater than 30. In some embodiments, m is less than 40, less than 35, less than 30, less than 25, or less than 20.

In some embodiments, the compound is selected from the group consisting of:

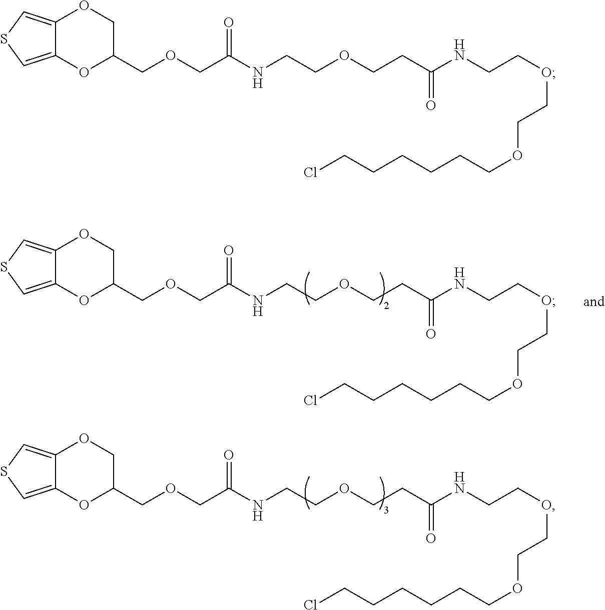

or a salt thereof.

### 3. Conductive Polymer Compositions

Also provided herein are electrically conductive compositions that can be used to provide an interface between an electrode and a specific cell or specific population of cells. Furthermore, the disclosed compositions can allow a cell or population of cells to be in electrical communication with the electrode, which can allow for the analysis and recording of neural circuits. The conductive polymer composition can include a polymer, e.g., a conductive polymer, and a dispersant. The polymer and the dispersant can interact through non-covalent interactions, such as ionic interactions. In addition, the composition can further include water.

The polymer and the dispersant can be included in the composition at varying amounts. For example, the composition can include the polymer and the dispersant at a concentration ratio of about 0.001:1 to about 10:1 (polymer:dispersant), such as about 0.01:1 to about 10:1, about 0.005:1 to about 1:1, about 0.01:1 to about 10:1, about 0.01:1 to about 5:1, about 0.01:1 to about 1:1, or about 0.001:1 to about 3:1.

a. Polymers

The polymer can be electrically conductive. The polymer can include polymerized compounds of formula (I). The polymer can include recurring units of formula (II)

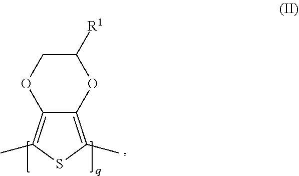

wherein: R^{a} is hydrogen or

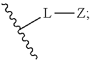

L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}— heteroalkylene-C(O)—, or heteroalkylene-OC(O)NR^{a}-heteroalkylene-C(O)—; R^{a} is hydrogen or alkyl; Z is a binding ligand selected from the group consisting of HaloTag, SNAP-tag, TMP-tag, βLac-tag, and CLIP-tag; and q is 1 to 10,000,000.

As the polymer can be synthesized from monomers of formula (I), the description of L and Z as described for formula (I) can be applied to recurring units of formula (II). In addition, the disclosed recurring units here and throughout can be randomly repeated throughout the polymer, in series, or a combination thereof.

In some embodiments, q is 1 to 5,000,000, 2 to 1,000,000, 2 to 9,000,000, 100 to 10,000,000, or 5 to 10,000,000. In some embodiments, q is greater than 1, greater than 5, greater than 10, greater than 50, greater than 100, greater than 200, greater than 500, greater than 1,000, or greater than 10,000. In some embodiments, q is less than 10,000,000, less than 8,000,000, less than 5,000,000, less than 1,000,000, or less than 500,000.

In some embodiments, the polymer includes recurring units of formula (III)

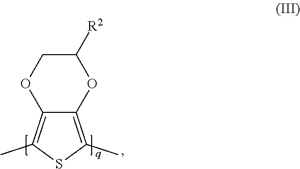

wherein: R² is hydrogen or X¹; X¹ is

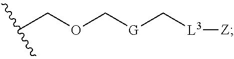

G is C(O)NR^{a} or C(O)O; R^{a} is hydrogen or C₁₋₃alkyl; L³ is (C—O—C)—C—C(O)—; Z is a binding ligand selected from the group consisting of HaloTag, SNAP-tag, TMP-tag, βLac-tag, and CLIP-tag; n is 1 to 40; and q is 1 to 10,000,000. The above description for q of formula (II) can also be applied to q for formula (II).

In some embodiments, the polymer is a copolymer. The copolymer can have a varying structure. For example, the copolymer can be a random copolymer, an alternating copolymer, or a block copolymer. The copolymer can include recurring units of formula (II-a)

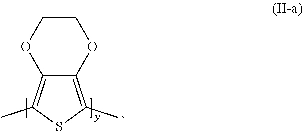

wherein y is 1 to 10,000,000; and recurring units of formula (II-b)

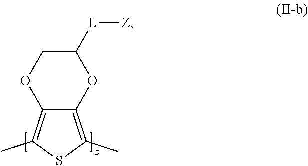

wherein z is 1 to 10,000,000. As the polymer can be synthesized from monomers of formula (I), the description of L and Z as defined for formula (I) can be applied to recurring units of formula (II-b).

In some embodiments, y is 1 to 5,000,000, 2 to 1,000,000, 2 to 9,000,000, 100 to 10,000,000, or 5 to 10,000,000, In some embodiments, y is greater than 1, greater than 5, greater than 10, greater than 50, greater than 100, greater than 200, greater than 500, greater than 1,000, or greater than 10,000. In some embodiments, y is less than 10,000,000, less than 8,000,000, less than 5,000,000, less than 1,000,000, or less than 500,000.

In some embodiments, z is 1 to 5,000,000, 2 to 1,000,000, 2 to 9,000,000, 100 to 10,000,000, or 5 to 10,000,000. In some embodiments, z is greater than 1, greater than 5, greater than 10, greater than 50, greater than 100, greater than 200, greater than 500, greater than 1,000, or greater than 10,000. In some embodiments, z is less than 10,000,000, less than 8,000,000, less than 5,000,000, less than 1,000,000, or less than 500,000.

In some embodiments, the copolymer includes recurring units of formula (II-a)

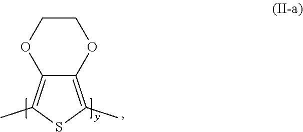

wherein y is 1 to 10,000,000; and recurring units of formula (II-c)

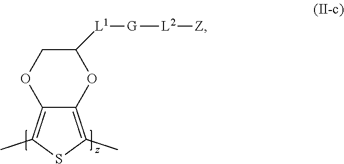

wherein z is 1 to 10,000,000 and L¹, L², G, and Z are defined as described above for formula (I-a). In addition, the above description for z of formula (II-b) can also be applied to z for formula (II-c).

In some embodiments, the copolymer includes recurring units of formula (II-a)

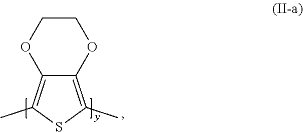

wherein y is 1 to 10,000,000; and recurring units of formula (III-a)

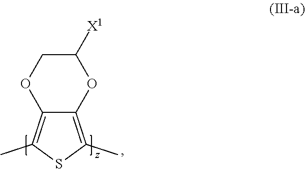

wherein z is 1 to 10,000,000 and X¹ is defined as described above for formula (III). The above description for z of formula (II-b) can also be applied to z for formula (III-a).

The polymer can be included in the composition at about 0.25% to about 90% by weight of the composition, such as about 0.5% to about 85% by weight of the composition, about 1% to about 80% by weight of the composition, about 2% to about 75% by weight of the composition, about 3% to about 60% by weight of the composition, about 4% to about 55% by weight of the composition, about 0.25% to about 50% by weight of the composition, about 0.5% to about 40% by weight of the composition, about 10% to about 90% by weight of the composition, or about 20% to about 85% by weight of the composition.

### i) Methods of Making Polymers

Further disclosed are methods of making the conductive polymers. The method can include electrochemically synthesizing the polymer. For example, the electrosynthesis of PEDOT:PSS is known in the art. However, it was found that when PSS was used as the dispersant for compounds of formula (I), polymerization was limited or did not occur. It is hypothesized, without being bound by any particular theory, that the -L-Z side chain creates steric interference with PSS, undermining polymerization. It was found, however, that this issue could be mitigated or overcome by using dispersants of lower molecular weight. For example, dispersants having a molecular weight of less than 1 kilodalton (kDa) could be used to allow for the electro-polymerization of the disclosed compounds of formula (I) into the disclosed polymers herein. In some embodiments, the dispersant has a molecular weight of less 0.9 kDa, less than 0.8 kDa, less than, 0.7 kDa, less than 0.6 kDa, less than 0.5 kDa, less than 0.4 kDa, less than 0.3 kDa, or less than 0.2 kDa. Further discussion of the dispersants can be found herein and its description can be applied to the disclosed methods of making the polymers.

The method can include adding a first mixture to an electrically conductive material, the first mixture comprising a solvent, a polystyrene sulfonate (PSS), and a 3,4-ethylenedioxythiophene (EDOT). The method can further include applying an electrical current to the electrically conductive material to provide a poly(3,4-ethylenedioxythiophene) (PEDOT) on a surface of the electrically conductive material. Accordingly, this step is similar to how PEDOT:PSS is done within the art.

The method can further include adding a second mixture to the electrically conductive material, the second mixture comprising a solvent, a dispersant having a molecular weight of less than 1 kDa, and a compound of formula (I), or a salt thereof. The method can further include applying an electrical current to the electrically conductive material to provide a second polymer attached to the PEDOT, wherein the second polymer includes recurring units of formula (l-b). It has been found that when synthesizing a first polymer of PEDOT and then polymerizing monomers of formula (I) off PEDOT, more stable compositions can be provided on the electrically conductive material. Formula (I) and formula (II-b) are defined above and its associated description can be applied to the methods of making the polymer.

The electrical current being applied can be any suitable range that allows the polymerization to proceed. In some embodiments, about 0.5 V to about 2.5 V can be applied to the electrically conductive substrate for polymerization of the monomer(s).

In some embodiments, the dispersant of the second mixture includes ClO₄⁻, BF₄⁻, PF₅⁻, BTFMSI, C₆H₅O₇⁻³, CO₃⁻², S₂O₃⁻², C₂H₃O₂⁻, HPO₄⁻², H₂PO₄⁻², Cl⁻, Br⁻, NO₃⁻, or a combination thereof. In some embodiments, the dispersant of the second mixture includes CIO₄, BF_{J}, or a combination thereof. The dispersant may be added as a monosalt. For example, the dispersant may be added as a monosalt with a counterion, such as Li, Na, K, or a combination thereof.

The solvent can be the same for the first mixture and the second mixture. However, in some embodiments, the solvent is different for the first mixture and the second mixture. In this latter embodiment, the solvents can be miscible with each other. The solvent may be aqueous. In some embodiments, the solvent is water.

The method may further include a washing step. For example, the provided conductive polymer (e.g., PEDOT-second polymer) attached to the electrically conductive substrate can be washed. The wash can be done with an alcohol. In some embodiments, the wash is done with ethanol.

The description of the monomers, polymers, dispersants, microelectrode devices, and electrically conductive materials can also be applied to the methods of making polymers disclosed herein.

b. Dispersant

The dispersant can be any suitable anion or counter ion that allows for the polymer to be electrochemically synthesized. Example dispersants include, but are not limited to, polystyrene sulfonate (PSS), ClO₄⁻, BF₄⁻, PF₆⁻, bis(trifluoromethylsulfonyl)imide (BTFMSI), C₆H₅O₇⁻³, CO₃⁻², S₂O₃⁻², C₂H₃O₂⁻, HPO₄⁻², H₂PO₄⁻, Cl⁻, Br⁻, NO₃⁻, and combinations thereof. In some embodiments, the dispersant includes PSS, ClO₄⁻, BF₄⁻ PF₆⁻, BTFMSI, C₆H₅O₇⁻³, CO₃⁻², S₂O₃⁻², C₂H₃O₂⁻, HPO₄⁻², H₂PO₄⁻, Cl⁻, Br⁻, NO₃⁻, or a combination thereof. In some embodiments, the dispersant includes PSS, LiClO₄, LiBF₄, or a combination thereof.

In addition to being a counter ion that can interact with the polymer, the dispersant may also be present in the composition as a monosalt. For example, the dispersant or a percentage of the dispersant may be present in the composition as a monosalt with a counterion, such as Li, Na, K, or a combination thereof

The dispersant can be included in the composition at about 0.5% to about 80% by weight of the composition, such as about 1% to about 75% by weight of the composition, about 2% to about 60% by weight of the composition, about 3% to about 55% by weight of the composition, about 5% to about 50% by weight of the composition, about 0.5% to about 50% by weight of the composition, about 0.5% to about 40% by weight of the composition, about 10% to about 75% by weight of the composition, or about 10% to about 80% by weight of the composition.

### 4. Microelectrode Arrays

Also disclosed herein are microelectrode arrays. The microelectrode arrays can be used in the disclosed methods, e.g. methods of detecting electrical activity in a cell. The microelectrode arrays may include an electrically conductive material. Any suitable material can be used for the electrically conductive material as long as it can provide access to the conductive element. Examples include, but are not limited to, gold, platinized-platinum, tungsten, platinum, platinum iridium, tantalum pentoxide, titanium nitride, and indium tin oxide. In some embodiments, the electrically conductive material is one or more of gold, platinized-platinum, tungsten, platinum, platinum iridium, tantalum pentoxide, titanium nitride, and indium tin oxide. In some embodiments, the electrically conductive material includes gold.

The microelectrode array may further include a conductive polymer composition, as disclosed herein, attached to a surface of the electrically conductive material. The conductive polymer composition can be attached (e.g., assembled) to the surface of the electrically conductive material via a voltage-mediated redox polymerization reaction. This can achieve a physically stable interface between the material and the polymer. The stability may arise from electrostatic and/or Van Der Waals forces. Accordingly, the conductive polymer composition can attach to the surface of the electrically conductive material through non-covalent interactions, such as electrostatic, Van Der Waals, hydrogen bonding, or a combination thereof. In other embodiments, assembly of the conductive polymer composition can be carried out through chemical redox methods.

### 5. Genetic Constructs

The conductive polymer composition or a component thereof may bind to a protein expressed on the surface of a cell. The protein expressed on a surface of a cell may be encoded by or comprised within a genetic construct, which is referred to as a “genetically encoded protein” herein. The genetically encoded protein may be one or more of HaloTag protein, SNAP-tag protein, TMP-tag protein, βLac-tag protein, and CLIP-tag protein. The genetic construct, such as a plasmid or expression vector, may comprise a nucleic acid that encodes a protein. In certain embodiments, a genetic construct may encode one genetically encoded protein, and optionally a marker protein. In some embodiments, a genetic construct may encode two genetically encoded proteins, i.e., a first genetically encoded protein and a second genetically encoded protein, and optionally a marker protein. In some embodiments, a first genetic construct may encode one genetically encoded protein, i.e., a first genetically encoded protein, and optionally a marker protein, and a second genetic construct may encode one genetically encoded protein, i.e., a second genetically encoded protein, and optionally a marker protein. In some embodiments, a first genetic construct may encode one genetically encoded protein, i.e., a first genetically encoded protein, and a second genetic construct may encode a marker protein. The marker protein may be one or more of lacZ (b-galactosidase), xylE (catechol 2,3-dioxygenase), lux (bacterial luciferase), luc (insect luciferase), phoA (alkaline phosphatase), gusA and gurA (beta-glucuronidase), GFP (green fluorescent protein), mCherry, dTomato, EGFP (Enhanced green fluorescent protein), DsRed (Discosoma sp. red fluorescent protein), Hygro (hygromycin), bla (beta-lactamase) and other antibiotic resistance markers, and the like.

Genetic constructs may include polynucleotides such as vectors and plasmids. The genetic construct may be a linear minichromosome including centromere, telomeres, or plasmids or cosmids. The vector may be an expression vector or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant rabies virus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise a heterologous nucleic acid encoding the genetically encoded protein and may further comprise an initiation codon, which may be upstream of the genetically encoded protein coding sequence, and a stop codon, which may be downstream of the genetically encoded protein coding sequence. The initiation and termination codon may be in frame with the genetically encoded protein coding sequence. The vector may also comprise a promoter that is operably linked to the genetically encoded protein coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a neuronal subtype-specific promoter. The tissue specific promoter may be a cardiomyocyte-specific promoter. The genetically encoded protein may be under the light-inducible or chemically inducible control to enable the dynamic control of expression of the genetically encoded protein in space and time. The promoter operably linked to the genetically encoded protein coding sequence may be a promoter any promoter known in the art. Examples of promoters include, but are not limited to, glial fibrillary acidic protein (GFAP), Tet-On, Tet-Off, simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, a Rous sarcoma virus (RSV) promoter, a CMV early enhancer/chicken P actin (sCAG) promoter, a human cytomegalovirus (hCMV) promoter, a mouse phosphoglycerate kinase (mPGK) promoter, or a human synapsin (hSYN) promoter.

The vector may also comprise a polyadenylation signal, which may be downstream of the genetically encoded protein coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal.

Coding sequences in the genetic construct may be optimized for stability and high levels of expression.

The genetic construct may also comprise an enhancer upstream of the genetically encoded protein coding sequence. The enhancer may be necessary for DNA expression. The enhancer may be any enhancer commonly used in the art. Examples of enhancers include, but are not limited to, human actin, human myosin, human hemoglobin, human muscle creatine, or a viral enhancer such as one from CMV, HA, RSV, or EBV. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered.

The genetic construct may be useful for transfecting cells with nucleic acid encoding the genetically encoded protein, where the transformed host cell may be cultured and maintained under conditions wherein expression of the genetically encoded protein takes place. The genetic construct may be transformed or transduced into a cell. The genetic construct may be formulated into any suitable type of delivery vehicle including, for example, a viral vector, lentiviral expression, electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a genetically encoded protein as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

a. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector may be a modified lentiviral vector. In some embodiments, the viral vector may be an adeno-associated virus (AAV) vector. The AAV vector is a small virus belonging to the genus Dependovirus of the Parvoviridae family that infects humans and some other primate species.

AAV vectors may be used to deliver the genetically encoded protein using various construct configurations. In some embodiments, the AAV vector may be a modified AAV vector. The modified AAV vector may have enhanced neuronal, cardiac muscle, and/or skeletal muscle tissue tropism. The modified AAV vector may be capable of delivering and expressing the genetically encoded protein in the cell of a mammal. The modified AAV vector may be based on one or more of several capsid types, including AAV1, AAV2, AAV5, AAV6, AAV8, and AAV9. The modified AAV vector may be based on an AAV pseudotype with alternative AAV capsids. For example, the AAV vector may be based on an AAV1, AAV6, or AAV7 pseudotype with alternative neuron-tropic AAV capsids, such as AAVrh10, AAV7m8, AAV2retro. In another example, the AAV vector may be based on an AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery.

### 6. Pharmaceutical Compositions

Further provided herein are pharmaceutical compositions comprising the above-described genetically encoded proteins. In some embodiments, the pharmaceutical composition may comprise about 1 ng to about 10 mg of DNA encoding the genetically encoded protein. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation may be used. Generally, additives for isotonicity may include sodium chloride, dextrose, mannitol, sorbitol, and lactose. In some cases, isotonic solutions such as phosphate buffered saline may be used. Stabilizers may include gelatin and albumin. A vasoconstriction agent may be added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analogs including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate.

### 7. Administration

The polymers, conductive polymer compositions, or microelectrode arrays as detailed herein, or at least one component thereof, may be administered or delivered to a cell. The polymers, conductive polymer compositions, or microelectrode arrays as detailed herein, or at least one component thereof, may be administered to a subject. The genetic constructs as described herein, or the pharmaceutical compositions comprising the same, may be administered or delivered to a cell. The genetic constructs as described herein, or the pharmaceutical compositions comprising the same, may be administered to a subject. Such compositions can be administered separately or together. Such compositions may be administered in dosages and by techniques well known to those skilled in the medical arts taking into consideration such factors as the age, sex, weight, and condition of the particular subject, and the route of administration.

The presently disclosed polymers, conductive polymer compositions, or microelectrode arrays as detailed herein, or at least one component thereof, and the genetic constructs as described herein, or the pharmaceutical compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular, or combinations thereof. The polymers, conductive polymer compositions, or microelectrode arrays as detailed herein, or at least one component thereof, may be delivered to a subject by several technologies including stereotactic injection, robotic implantation (e.g., Neuralink, Neuralink Corporation, San Francisco, CA), and the like. The genetic constructs as described herein, or the pharmaceutical compositions comprising the same, may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. Any of the compositions may be injected into the brain or other component of the central nervous system. Any of the compositions may be injected into the skeletal muscle or cardiac muscle. For veterinary use, polymers, conductive polymer compositions, or microelectrode arrays as detailed herein, or at least one component thereof, and the genetic constructs as described herein, or the pharmaceutical compositions comprising the same, may be administered as a suitably acceptable formulation in accordance with normal veterinary practice. The veterinarian may readily determine the dosing regimen and route of administration that is most appropriate for a particular animal. The polymers, conductive polymer compositions, or microelectrode arrays as detailed herein, or at least one component thereof, and the genetic constructs as described herein, or the pharmaceutical compositions comprising the same, may be administered by traditional syringes, needleless injection devices, “microprojectile bombardment gone guns,” or other physical methods such as electroporation (“EP”), “hydrodynamic method”, intravenous injection, retro-orbital injection, or ultrasound.

Upon delivery of the presently disclosed the genetic constructs as described herein, or the pharmaceutical compositions comprising the same, into the cells of a subject, the cells may express the genetically encoded protein on the surface of the cells. Upon delivery of the presently disclosed polymers, conductive polymer compositions, or microelectrode arrays as detailed herein, or at least one component thereof, into a subject, the polymers comprising a ligand for the genetically encoded protein may bind to the cells expressing the genetically encoded protein.

a. Cell Types

Any of the delivery methods and/or routes of administration detailed herein can be utilized with a myriad of cell types, for example, those cell types currently under investigation for electrophysiological recordings such as those capable of depolarizing. These cell types include, but not limited to, neurons, muscle cells (e.g., skeletal, cardiac, and smooth), endocrine cells (e.g., insulin-releasing pancreatic P cells), keratinocytes, glia, cell lines expressing voltage-gated ion channels.

### 8. Methods of Detecting Electrical Activity in a Cell

Provided herein are methods of detecting electrical activity in a cell. The methods may include contacting a microelectrode array as described herein with a cell and detecting electrical activity in the cell by covalently attaching the polymer of the conductive polymer composition to a protein on the surface of the cell. Detecting electrical activity in a cell may comprise measuring a change in voltage, a change in current, or a combination thereof. The change in voltage, the change in current, or a combination thereof may be used for decoding neural connectivity, frequency of firing, or a combination thereof. The cell may be any cell described herein. The microelectrode array may be implanted into a subject using the administration methods described herein. The protein on the surface of the cell may be a genetically encoded protein. The genetically encoded protein may be delivered to the cell with a virus as described herein. The protein on the surface of the cell may be one or more of HaloTag protein, SNAP-tag protein, TMP-tag protein, βLac-tag protein, and CLIP-tag protein.

### 9. Kits

Also provided herein are kits, which may be used to express a genetically encoded protein in cells of interest and detect electrical activity in those cells. The kits may include one or more of the monomers, the polymers, the microelectrode arrays, compositions and devices comprising the polymers or the microelectrode arrays, and means for expressing a protein in a cell and for detecting electrical activity in cells, as described above.

The kits also may include instructions for using the components included in the kits. Instructions included in kits may be affixed to packaging material or may be included as a package insert. While the instructions are typically written on printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### 10. EXAMPLES

The foregoing may be better understood by reference to the following examples, which are presented for purposes of illustration and are not intended to limit the scope of the disclosure. The present disclosure has multiple aspects and embodiments, illustrated by the appended non-limiting examples.

### Example 1

### Monomer Synthesis

All chemical reagents were obtained from commercial suppliers and used without further purification unless otherwise stated. Anhydrous solvents were purchased from Sigma-Aldrich, and dried over 3 Å molecular sieves when necessary. Normal-phase flash column chromatography was performed using Biotage KP-Sil 50 μm silica gel columns and ACS grade solvents on a Biotage Isolera flash purification system or Teledyne Combiflash using Silicycle silica gel columns. Reverse phase preparative HPLC was performed with the following conditions: Phenomenex Kinetex C18, 50×30 mm; 5 μm. Eluting with a gradient of acetonitrile:water specified for each compound with 0.1% formic acid over 5 min at a flow rate of 50 mL/min. Proton (1H), and carbon (13C) NMR spectra were recorded on a 500 MHz Bruker Avance Ill with direct cryoprobe spectrometer. Chemical shifts were reported in ppm (δ) and were referenced using residual nondeuterated solvent as an internal standard (CDCl₃ at 7.24 ppm for 1H-NMR and 77.0 for 13C-NMFR. CD₃OD at 3.33 ppm for 1H-NMR and 47.6 for 13C-NMR. DMSO-d₅ at 2.52 ppm for 1H-NMR and 39.9 ppm for 13C-NMR). Proton coupling constants are expressed in hertz (Hz). The following abbreviations were used to denote spin multiplicity for proton NMR: s=singlet, d=doublet, t=triplet, q=quartet, m=multiplet, br.s=broad singlet, dd=doublet of doublets, dt=doublet of triplets, quin=quintet, tt=triplet of triplets. Low resolution liquid chromatography/mass spectrometry (LCMS) was performed on a Waters Acquity-H UPLC/MS system with a 2.1 mm×50 mm, 1.7 μm, reversed phase BEH C18 column and LCMS grade solvents. A gradient elution from 95% water+0.1% formic acid/5% acetonitrile+0.10% formic acid to 95% acetonitrile+0.1% formic acid/5% water+0.1% formic acid over 2 min plus a further minute continuing this mixture at a flow rate of 0.85 mL/min was used as the eluent. Total ion current traces were obtained for electrospray positive and negative ionization (ESI+/ESI−). All IUPAC compound names were generated using Chemdraw Version 19.1.1.21.

### Synthesis of N-(2-(2-((6-chlorohexyl)oxy)ethoxy)ethyl)-3-((1-(2,3-dihydrothieno[3,4-b][1,4]dioxin-2-yl)-4-oxo-2,8,11-trioxa-5-azatridecan-13-yl)oxy)propenamide

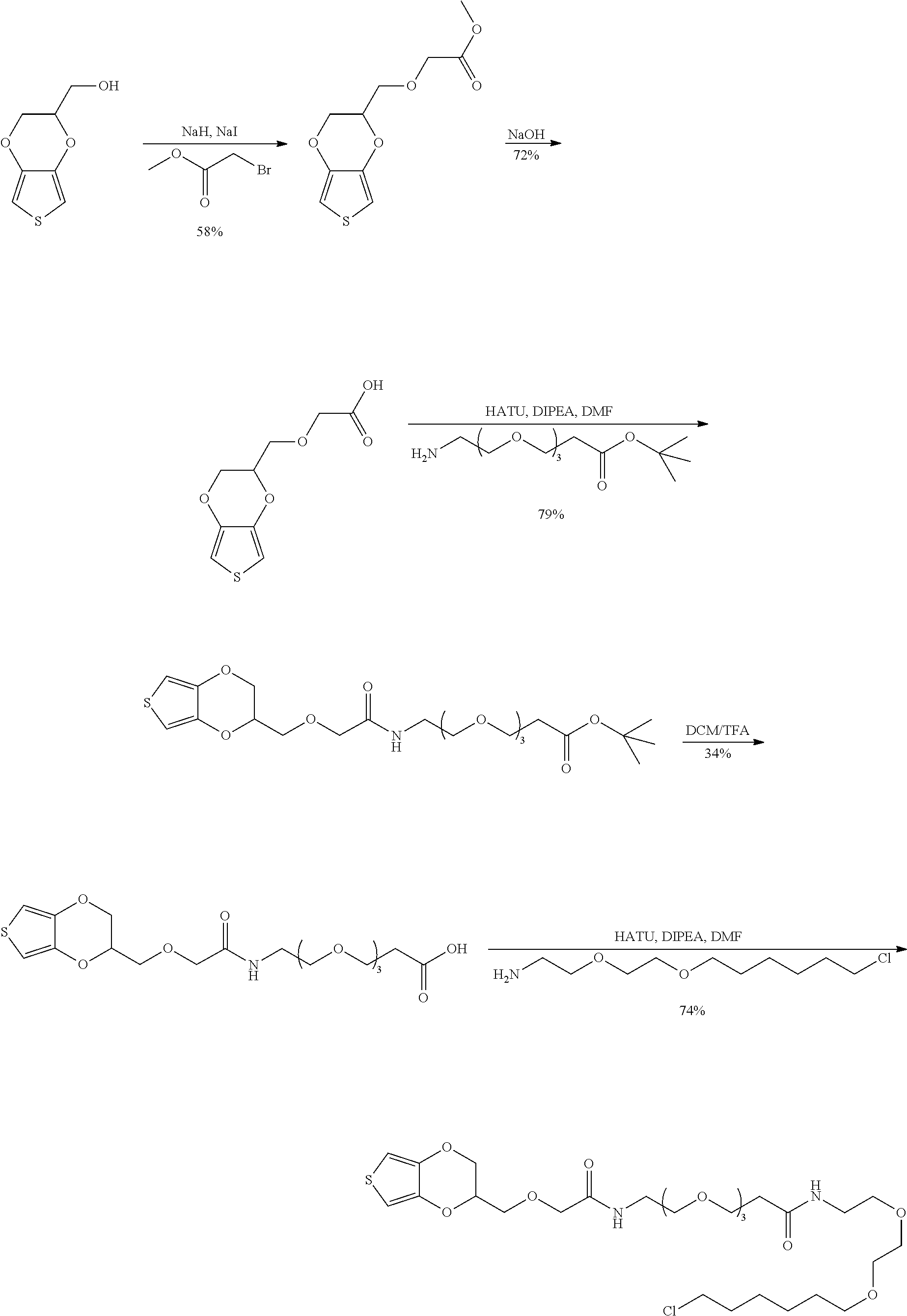

### Step 1: Synthesis of methyl 2-(2,3-dihydrothieno[3,4-b][1,4]dioxin-2-yl)methoxy)acetate

To a round bottom flask was added sodium iodide (0.17 g, 0.2 Eq, 1.2 mmol), NaH (0.28 g, 60% Wt, 1.2 Eq, 7.0 mmol), and THF (12 mL). The mixture was cooled in an ice/water bath and placed under nitrogen after which a solution of (2,3-dihydrothieno[3,4-b][1,4]dioxin-2-yl)methanol (1.0 g, 1 Eq, 5.8 mmol) in THF (12 mL) was added dropwise. After 15 min, methyl bromoacetate (1.1 g, 0.64 mL, 1.2 Eq, 7.0 mmol) in THF (3 mL) was added dropwise and the reaction was left to stir overnight after which LCMS indicated product.

The reaction was quenched with water (5 mL) and diluted with ethyl acetate (40 mL). After separation of layers, the aqueous layer was extracted with ethyl acetate (20 mL). Combined organic extracts were washed with brine (10 mL), filtered through an isolute phase separator, and concentrated to an oil which was purified by silica gel chromatography using a gradient of 0 to 50% ethyl acetate in hexanes to give methyl 2-((2,3-dihydrothieno[3,4-b][1,4]dioxin-2-yl)methoxy)acetate (818 mg, 3.35 mmol, 58%) as a yellow oil. ¹H NMR (500 MHz, CDCls) δ 6.32 (d, J 3.7 Hz, 1H), 6.31 (d, J=3.7 Hz, 1H), 6.28 (d, J=3.3 Hz, 1H), 4.35 (dtd, J=7.5, 5.3, 2.3 Hz, 1H), 4.26 (dd, J=11.7, 2.3 Hz, 1H), 4.16 (s, 2H), 4.09 (dd, J=11.7, 7.4 Hz, 1H), 3.82 (dd, J=10.4, 5.1 Hz, 1H), 3.77-3.75 (m, 1H), 3.74 (s, 3H). For a literature synthesis of this compound, see: Langmuir. 2008, 24, 8071-8077, which is incorporated by reference herein in its entirety.

### Step 2: Synthesis of 2-(2,3-dihydrothieno[3,4-b][1,4]dioxin-2-yl)methoxy)acetic acid

To a round bottom flask containing methyl 2-((2,3-dihydrothieno[3,4-b][1,4]dioxin-2-yl)methoxy)acetate (818 mg, 1.0 Eq, 3.35 mmol) in THF (4 mL) was added NaOH (4 M) (1.21 g, 7.5 mL, 4.0 molar, 9 Eq, 30.1 mmol) and the reaction was stirred at room temperature for 1 h after which THF was removed on the rotary evaporator and HCl (6 M) (1.10 g, 5.0 mL, 6.0 molar, 9 Eq, 30.1 mmol) was added. Additional water was added and the mixture was stirred vigorously for 20 min then filtered and dried to give 2-((2,3-dihydrothieno[3,4-b][1,4]dioxin-2-yl)methoxy)acetic acid (557 mg, 2.42 mmol, 72.2%) as a light gray solid. ¹H NMR (500 MHz, CDCl₃) δ 6.34 (d, J=3.7 Hz, 1H), 6.32 (d, J=3.7 Hz, 1H), 4.36 (dtd, J=7.4, 5.1, 2.3 Hz, 1H), 4.24 (dd, J=11.7, 2.3 Hz, 1H), 4.09 (dd, J=11.8, 7.4 Hz, 1H), 3.84 (dd, J=10.6, 5.2 Hz, 1H), 3.79 (dd, J=10.6, 5.1 Hz, 1H). For a literature synthesis of this compound, see: Langmuir. 2008, 24, 8071-8077, which is incorporated by reference herein in its entirety.

### Step 3: Synthesis of tert-butyl 1-(2,3-dihydrothieno[3,4-b][1,4]dioxin-2-yl)-4-oxo-2,8,11,14-tetraoxa-5-azaheptadecan-17-oate

To a vial containing 2-((2,3-dihydrothieno[3,4-b][1,4]dioxin-2-yl)methoxy)acetic acid (338 mg, 1 Eq, 1.47 mmol) was added DMF (2 mL) followed by DIPEA (380 mg, 0.51 mL, 2 Eq, 2.94 mmcl) and HATU (837 mg, 1.5 Eq, 2.20 mmol). The mixture was stirred for 10 min after which tert-butyl 3-(2-(2-(2-aminoethoxy)ethoxy)ethoxy)propanoate (407 mg, 1 Eq, 1.47 mmol) in DMF (1 mL) was added and the reaction mixture was stirred at room temperature for 1 h. The reaction mixture was directly purified by RP HPLC eluting with 20 to 80 acetonitrile in water to give tert-butyl 1-(2,3-dihydrothieno[3,4-b][1,4]dioxin-2-yl)-4-oxo-2,8,11,14-tetraoxa-5-azaheptadecan-17-oate (570 mg, 1.16 mmol, 79.3%) as a yellow oil. (ES-LCMS) m/z 490.4 (M+H)+.

### Step 4: Synthesis of 1-(2,3-dihydrothieno[3,4-b][1,4]dioxin-2-yl)-4-oxo-2,8,11,14-tetraoxa-5-azaheptadecan-17-oic acid

To a round bottom flask containing tert-butyl 1-(2,3-dihydrothieno[3,4-b][1,4]dioxin-2-yl)-4-oxo-2,8,11,14-tetraoxa-5-azaheptadecan-17-oate (570 mg, 1 Eq, 1.16 mmol) was added DCM (7 mL) and the mixture was cooled in an ice/water bath after which TFA (1.53 g, 1.02 mL, 11.5 Eq, 13.4 mmol) was added slowly. The reaction mixture was removed from the ice bath and stirred at room temperature overnight after which it was concentrated and purified by RP HPLC eluting with 20 to 80% acetonitrile in water to give 1-(2,3-dihydrothieno[3,4-b][1,4]dioxin-2-yl)-4-oxo-2,8,11,14-tetraoxa-5-azaheptadecan-17-oic acid (172 mg, 397 μmol, 34.1%) as a brown oil. 1H NMR (500 MHz, CDC3) δ 7.12 (d, J=6.1 Hz, 1H), 6.36 (d, J=3.7 Hz, 1H), 6.32 (d, J=3.7 Hz, 1H), 4.34 (dtd, J=7.4, 5.0, 2.3 Hz, 1H), 4.22 (dd, J=11.7, 2.3 Hz, 1H), 4.09-4.06 (m, 1H), 4.05 (s, 2H), 3.74 (dt, J=12.0, 5.5 Hz, 4H), 3.64-3.53 (m, 11H), 3.53-3.44 (m, 2H), 2.58 (t, J=6.0 Hz, 2H).

Step 5: Synthesis of N-(2-(2-((6-chlorohexyl)oxy)ethoxy)ethyl)-3-((1-(2,3-dihydrothieno[3,4-b][1,4]dioxin-2-yl)-4-oxo-2,8,11-trioxa-5-azatridecan-13-yl)oxy)propenamide2-2-((6-chlorohexyl)oxy)ethoxy)ethan-1-amine was synthesized as described here: Nat Chem. Bio, 2011, 7, 538-543, which is incorporated by reference herein in its entirety

To a vial containing 1-(2,3-dihydrothieno[3,4-b][1,4]dioxin-2-yl)-4-oxo-2,8,11,14-tetraoxa-5-azaheptadecan-17-oic acid (150 mg, 1 Eq, 346 μmol) in DMF (1 mL) in an ice/water bath was added HATU (197 mg, 1.5 Eq, 519 μmol) and DIPEA (89.5 mg, 121 μL, 2 Eq, 692 μmol) after which the mixture was removed from the ice bath and stirred for 10 min. Then, the mixture was cooled in an ice/water bath and 2-(2-((6-chlorohexyl)oxy)ethoxy)ethan-1-amine (116 mg, 1.5 Eq, 519 μmol) in DMF (1 mL) was added and the reaction mixture was removed from the ice/water bath and stirred at room temperature for 1 h after which the reaction mixture was directly purified by RP HPLC eluting with 15 to 90% acetonitrile in water to give N-(2-(2-((6-chlorohexyl)oxy)ethoxy)ethyl)-3-((1-(2,3-dihydrothieno[3,4-b][1,4]dioxin-2-yl)-4-oxo-2,8,11-trioxa-5-azatridecan-13-yl)oxy)propanamide (164 mg, 257 μmol, 74.1%) as a brown oil. ¹H NMR (500 MHz, CDCl₃) δ 6.98 (t, J=5.9 Hz, 1H), 6.56 (q, J=4.5, 3.2 Hz, 1H), 6.36 (d, J=3.7 Hz, 1H), 6.33 (d, J=3.6 Hz, 1H), 4.33 (dtd, J=7.3, 5.0, 2.3 Hz, 1H), 4.22 (dd, J=11.7, 2.3 Hz, 1H), 4.06 (dd, J=11.7, 7.3 Hz, 1H), 4.03 (s, 2H), 3.79-3.67 (m, 4H), 3.62-3.40 (m, 27H), 2.44 (t, J=6.0 Hz, 2H), 1.93 (s, 1H), 175 (dt, J=14.6, 6.8 Hz, 2H), 1.63-1.51 (m, 2H), 1.43 (ddt, J=9.0, 7.1, 5.8 Hz, 2H), 1.38-1.30 (m, 2H).

### Example 2

PEDOT-HTL Conductive Polymer

To endow electrodes with covalent functionality, EDOT (3,4-ethylenedioxythiophene), a monomer known for its ability to decrease impedance by electro-polymerizing into a conductive PEDOT (poly-EDOT) polymer was focused on. Novel derivatives of EDOT containing the HaloTag Ligand (HTL)—a chemical moiety that spontaneously forms a covalent bond with the genetically encoded HaloTag Protein (HTP) was developed. The choice of the HTL/HTP system is based on DART (Drugs Acutely Restricted by Tethering), in which drug-HTL chemicals were delivered to neurons programmed to express HTP on their surface, producing localized pharmacology (Shields et al., Science. 2017; 356(6333):eaaj2161). The systems and methods provided herein covalently attach chemical matter to genetically defined neurons, with the goal of pioneering a new genetically encoded electrophysiology platform featuring a simple PEDOT-HTL conductive polymer, A wide range of PEDOT-HTL designs will be explored and binding capacity optimized with a custom biochemical assay.

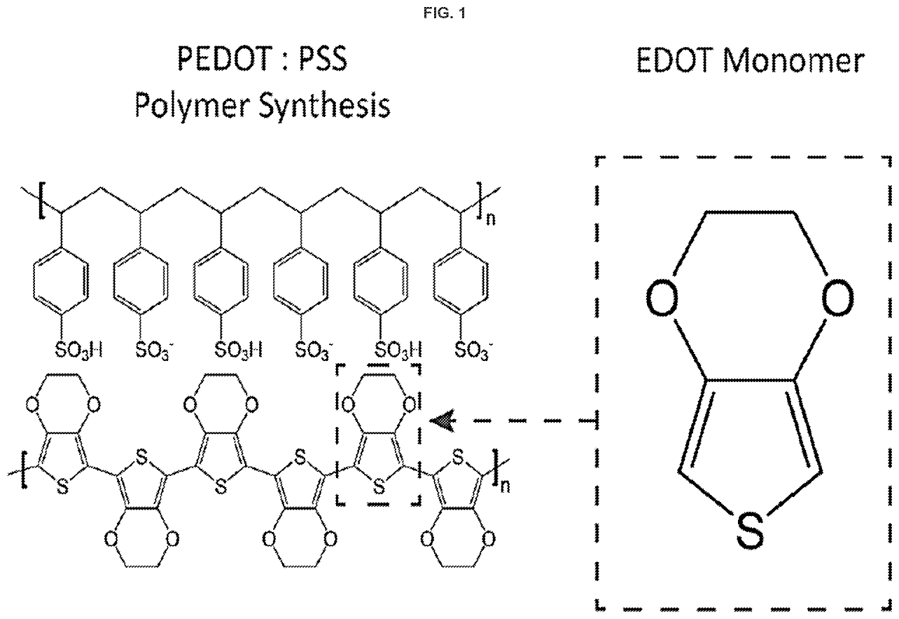

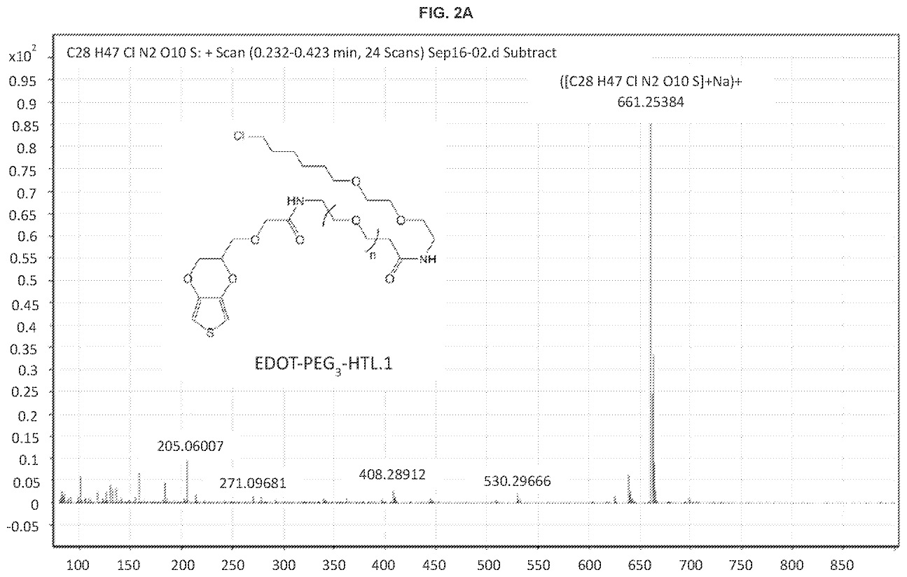

EDOT monomers can electro-polymerize readily into PEDOT polymer, particularly when the electrochemical reaction is performed in the presence of counter-ions, the most established of which is polystyrene sulfonate (PSS) (Láng et al., 2016, Zeitschrift Fur Physikalische Chemie, 230(9), 1281-1302; Schweiss et al., 2005, Eiectrochimica Acta, 50(14), 2849-2856). During the polymerization process, EDOT monomers (FIG. 1, insert) become covalently attached to one another via the electrochemical redox reaction (FIG. 1, bottom polymer). In contrast to the EDOT→PEDOT transformation, PSS begins as a macromolecule (FIG. 1, top macromolecule) and it is not covalently altered by the electrochemical process. PSS provides charge neutralization, and associates with the growing PEDOT polymer via hydrogen bonding, akin to the polar interactions that hold together complementary strands of DNA.

The goal was to develop a functionalized EDOT monomer, named EDOT^{HTL}, which incorporates the binding ligand (HTL) as an R-chain while retaining ability to electropolymerize at sufficient density and physical accessibility to enable attachment to HTP-expressing neurons. The approach was non-trivial for several reasons. In particular, EDOT conjugates containing an R-chain modification have been attempted by several groups, however, the majority have precluded electro-polymerization, likely due to sidechain steric interference and/or changes to other physiochemical properties of EDOT (Goll et al., 2015, Beilstein Journal of Organic Chemistry, 335-347; Hailemichael Ayalew et al., 2019, Polymers). An additional and major unknown, even if polymerization could be achieved with R-chain modified EDOT^{HTL} is the lack of precedent with which to estimate the polymer density and topology needed to coax an HTP-expressing neuron to invade the polymer and bind to the embedded HTL moieties.

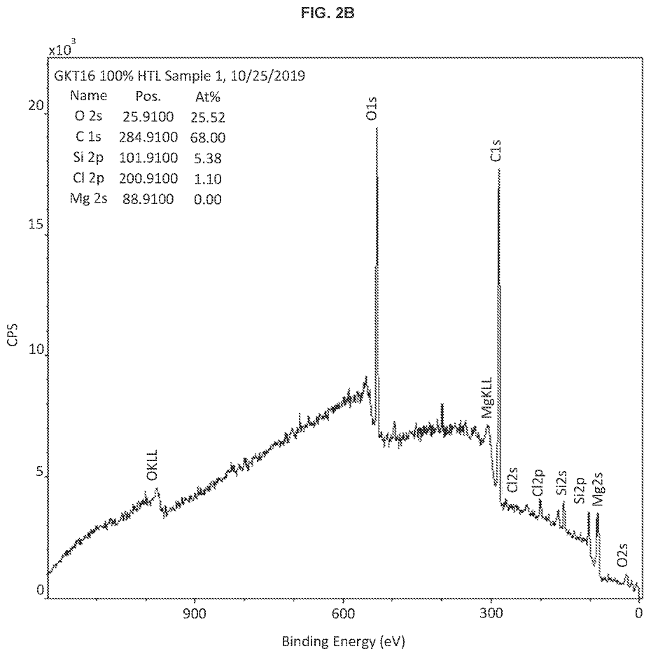

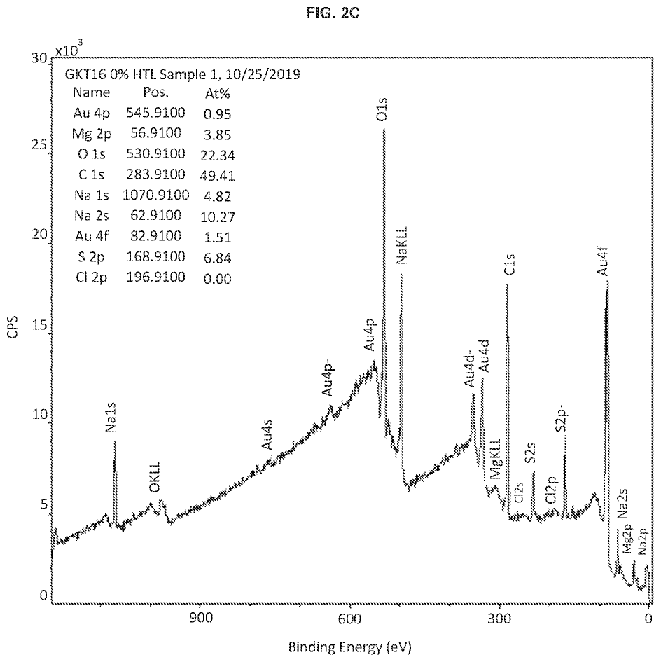

EDOT conjugates with HTL have not been reported previously. Initially a series of EDOT^{PEGn-HTL} monomers (n=1,2,3, . . . ) were synthesized, wherein it was anticipated that increasing the number of PEG repeats would improve solubility, at the risk of exacerbating sidechain steric interference (FIG. 2A). The HTL modification increases the molecular weight of the monomer. The unmodified EDOT has a molecular weight (MW) of 42; while EDOT^{PEGn-HTL} has a MW of 551 (n=1), 595 (n=2), 639 (n=3), or MW of 507+44×n. Thus, all of the candidate designs carry substantial steric bulk, which is inevitable.

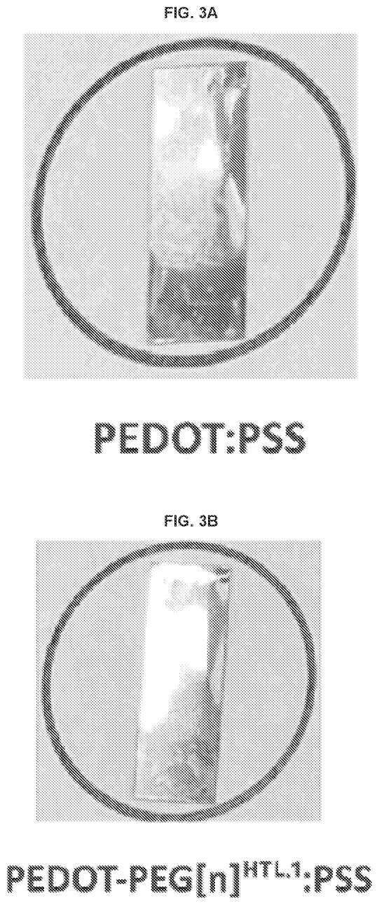

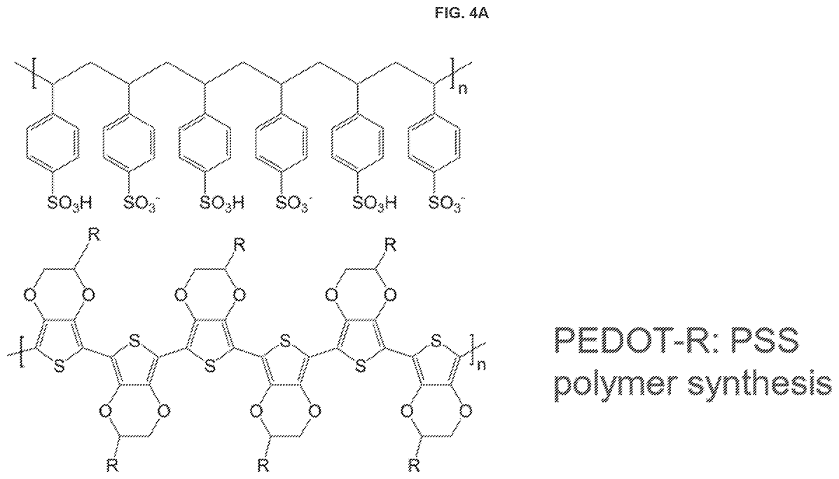

Several conditions were tested, including various solvents, against the three variants. It was found that EDOT^{PEG3-HTL} was the most soluble. EDOT^{PEG3-HTL} was unable to be polymerized with PSS as the counter-ion. Specifically, whereas the formation of PEDOT:PSS is accompanied by an unmistakable darkening of a gold surface (i.e., the polymer is visible to the naked eye), no visual indication of polymerization was seen when EDOT was replaced with EDOT^{PEG3-HTL} (FIG. 3A and FIG. 3B). This held true over a wide range of voltage parameters and concentrations.

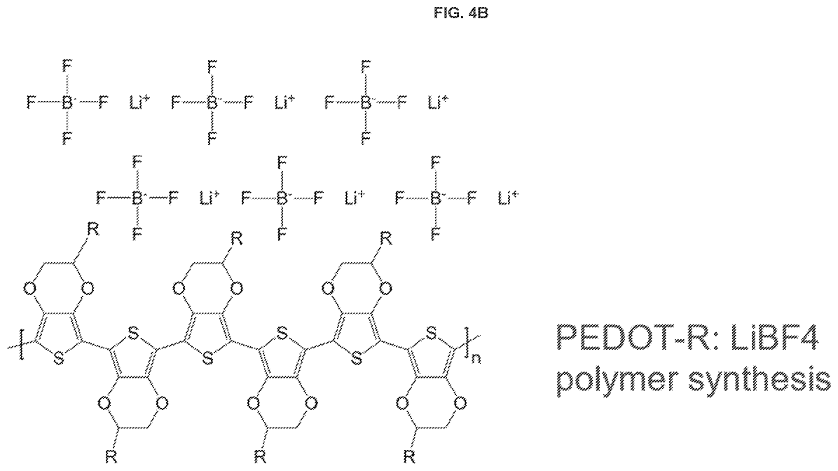

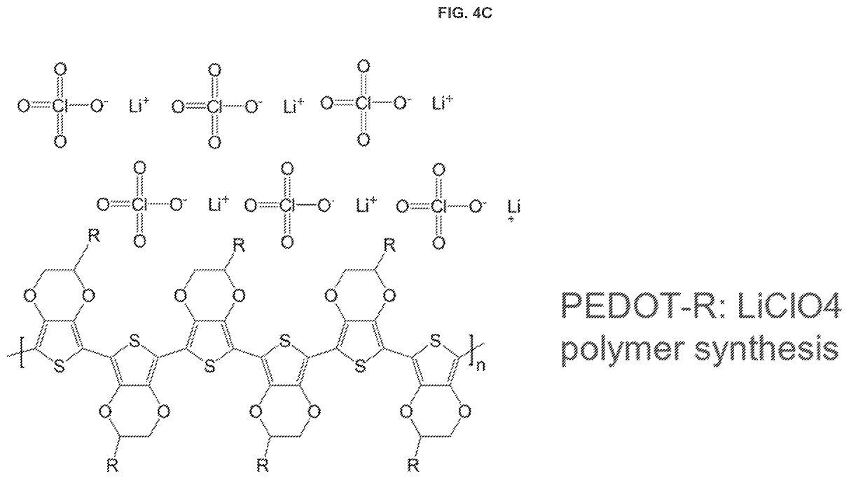

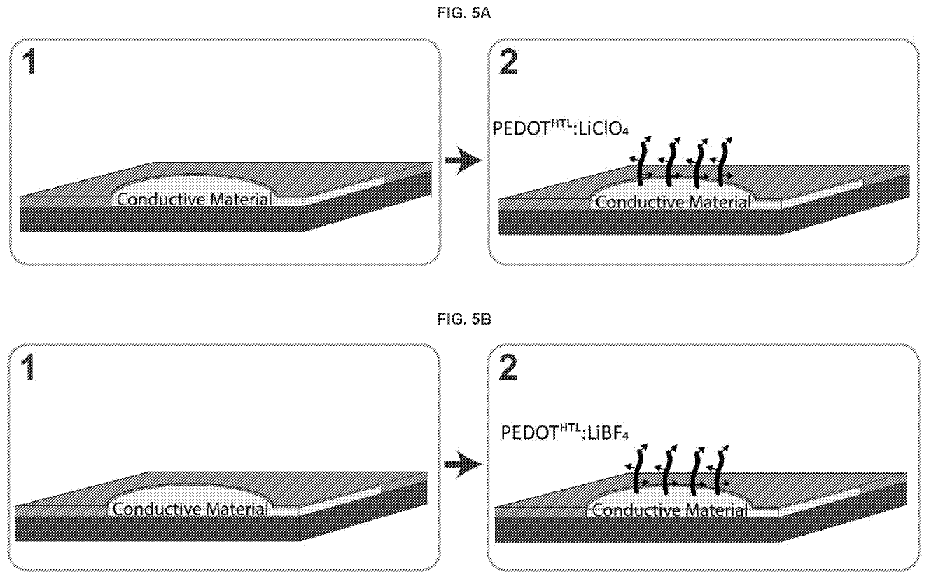

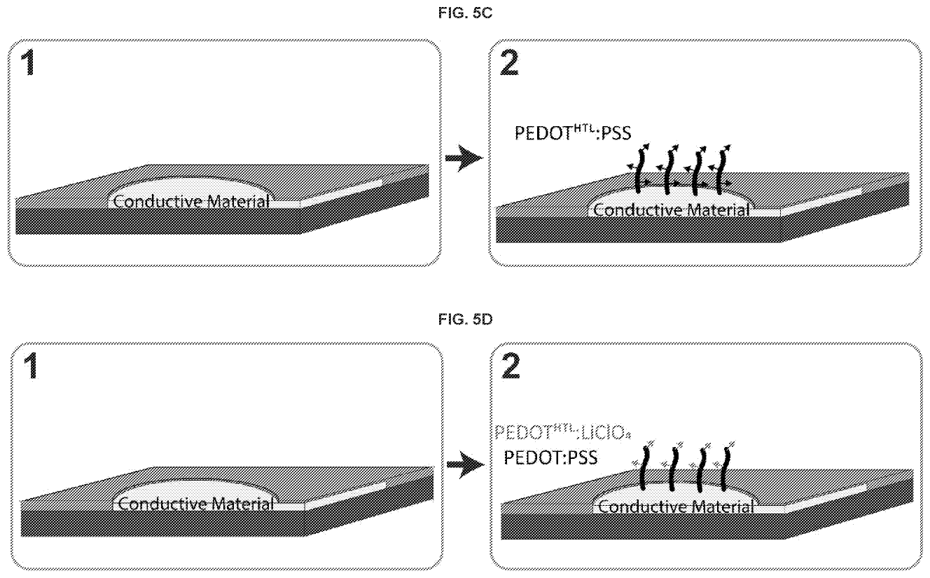

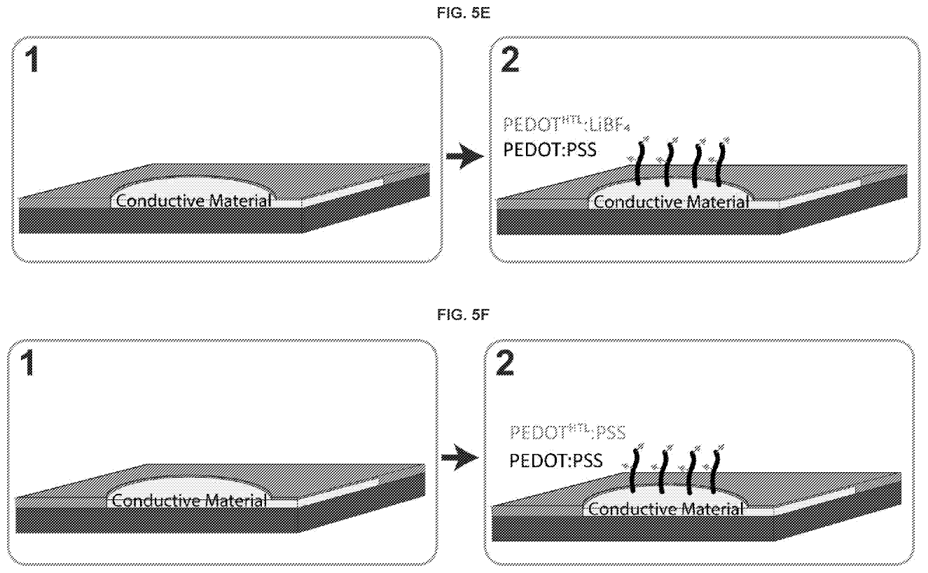

It was hypothesized that the issue was that of sidechain steric interference, given the packed structure of PEDOT:PSS (FIG. 1). Next, alternative counter ions to PSS were tested, reasoning that the macromolecular nature of PSS would lack the ability to conform around a large R group. Perchlorate (ClO₄⁻) and tetrafluoroborate (LiBF₄⁻) were selected as promising candidates. Using a 1-step polymerization process (at various voltages ranging 0.5 V to 2 V) PEDOT^{PEG3-HTL}:LiBF₄ and PEDOT^{PEG3-HTL}:LiClO₄ were readily polymerized (FIG. 4C and FIGS. 5A-F).

Although electrochemically successful, it was observed that polymers featuring small counterions lacked the physical durability of PEDOT:PSS. Specifically, it was observed that both of these polymer coatings would disintegrate into fine black dust when exposed to ethanol or following more than 12 hours in an aqueous environment. It was hypothesized that the instability of these polymers was due to that small counter ions (LiBF₄ and LiClO₄) did not provide the backbone scaffold afforded by PSS. Thus, combining the two in a co-polymerization scheme seen in FIGS. 5A-F was tested. Titrating mixtures of EDOT^{HTL} with its smaller EDOT cousin could allow assembly of longer polymer chains, albeit lowering the fraction of HTL-containing units. In such permutations, the goal is to maximize the number of HTLs on the conductive surface.

A novel two-stage polymerization procedure was also developed, in which a layer of PEDOT:PSS was first deposited, and followed by PEDOT^{PEG3-HTL}:ClO₄⁻ or PEDOT^{PEG3-HTL}:BF₄⁻. This two-step polymerization proved to be the most effective at combining PEDOT^{PEG3-HTL} into the polymer. In particular, the design afforded the physical stability of the PEDOT:PSS base layer, along with the steric compatibility with the bulkier PEDOT^{PEG3-HTL}. Unlike the monolayer, it was found that the two-layer design was robust to physical perturbation, including ethanol wash and extended time in aqueous conditions. As an additional surprise, it was found that an ethanol wash improved HTL accessibility, through a process termed herein as “ethanol thinning” of the upper layer. It is believed that this process reflects the covalent interaction between the structurally stable PEDOT:PSS base layer, and a subset of the PEDOT^{PEG3-HTL} strands. Such a covalent attachment would not be possible in the monolayer approach. Thus, it is believed that the two-layer approach is a fundamental design advance.

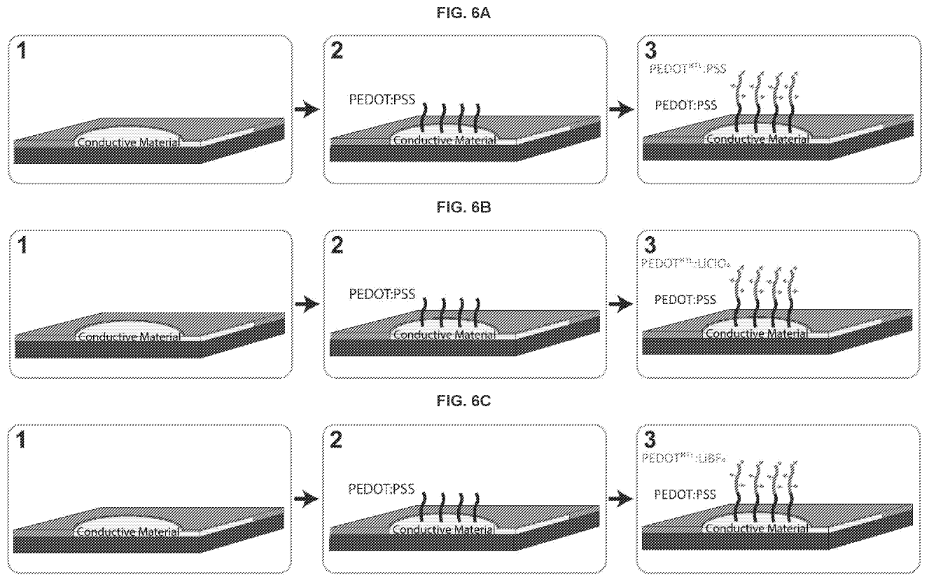

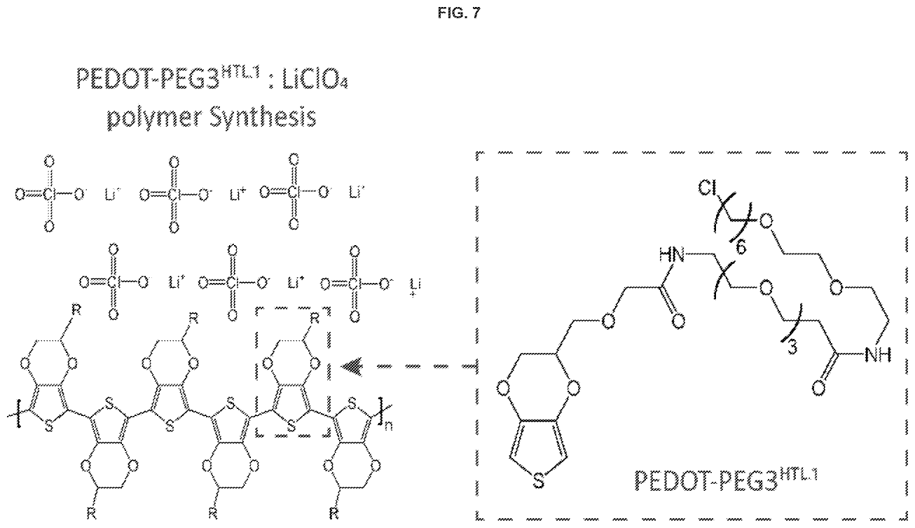

A successful polymerization strategy was identified, which employs the two step polymerization method with PEDOT:PSS as the base layer and PEDOT^{PEG3-HTL}:LiClO₄ as the second layer. A diagram of this successful polymerization is shown in FIG. 6B, with the chemical components shown in FIG. 7. The best binding of this variant occurred after the fully assembled polymer was washed with ethanol, which removed any non-polymerized monomer from the polymer, maximizing the area for binding availability.

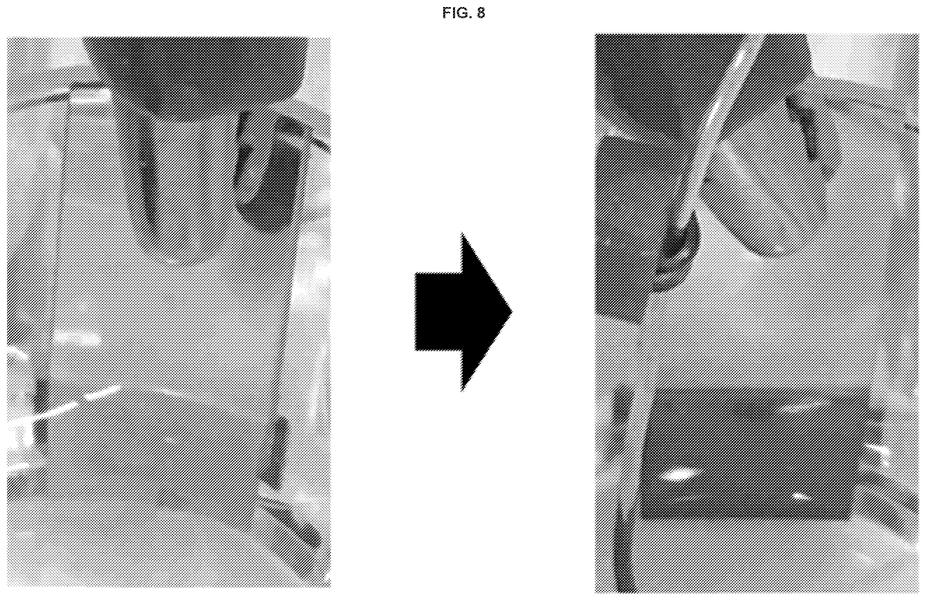

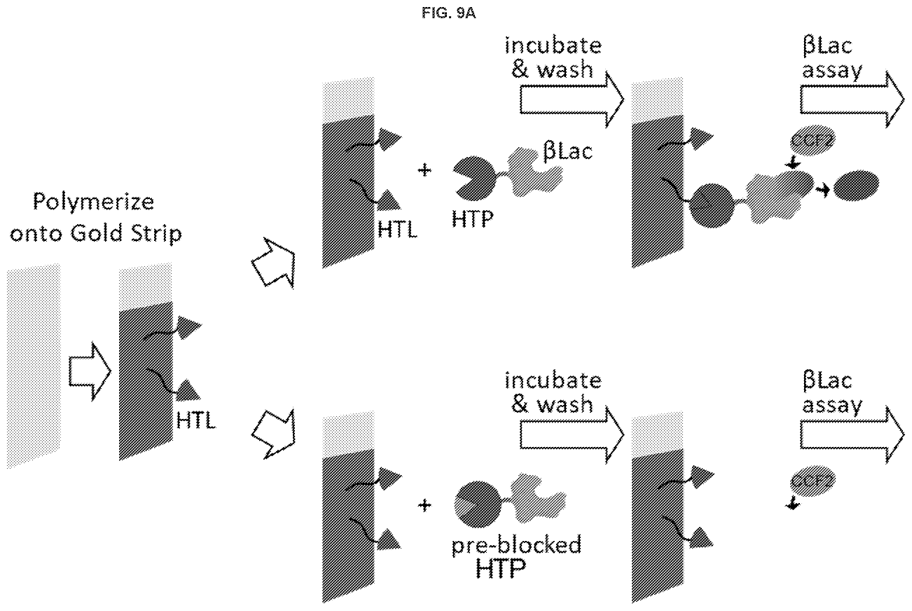

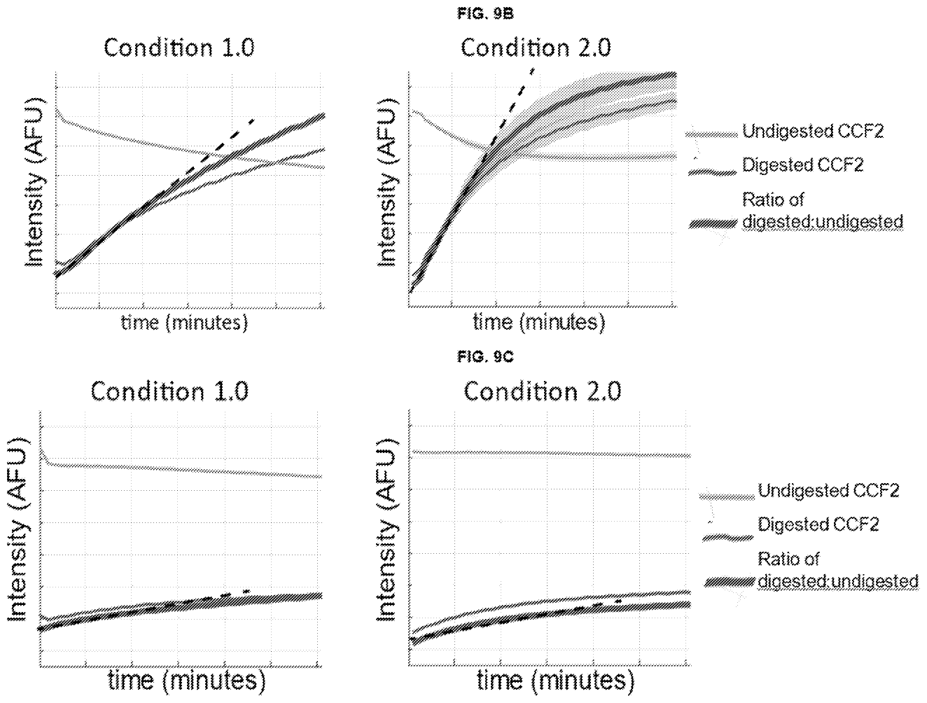

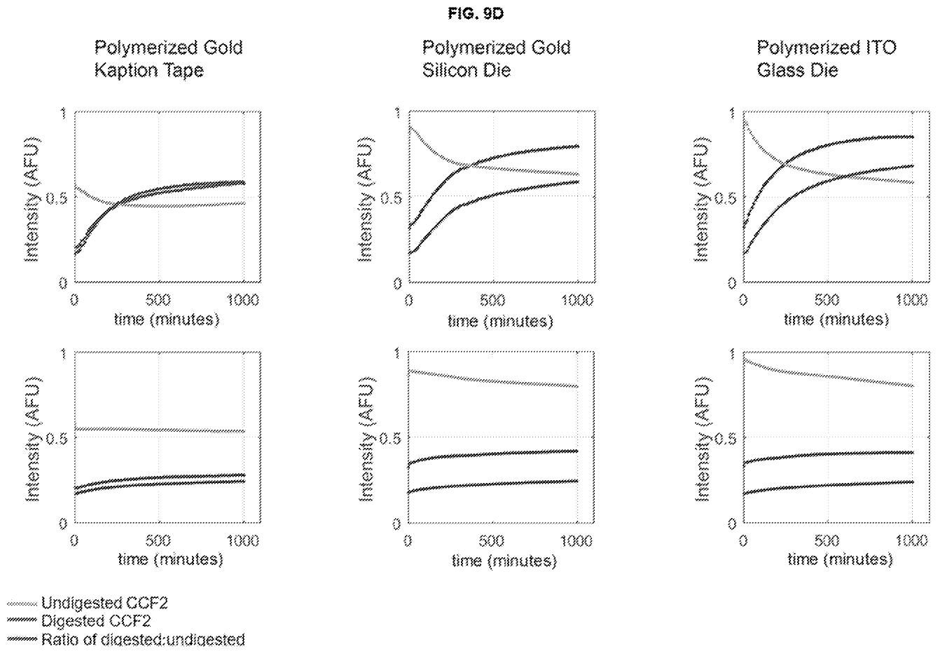

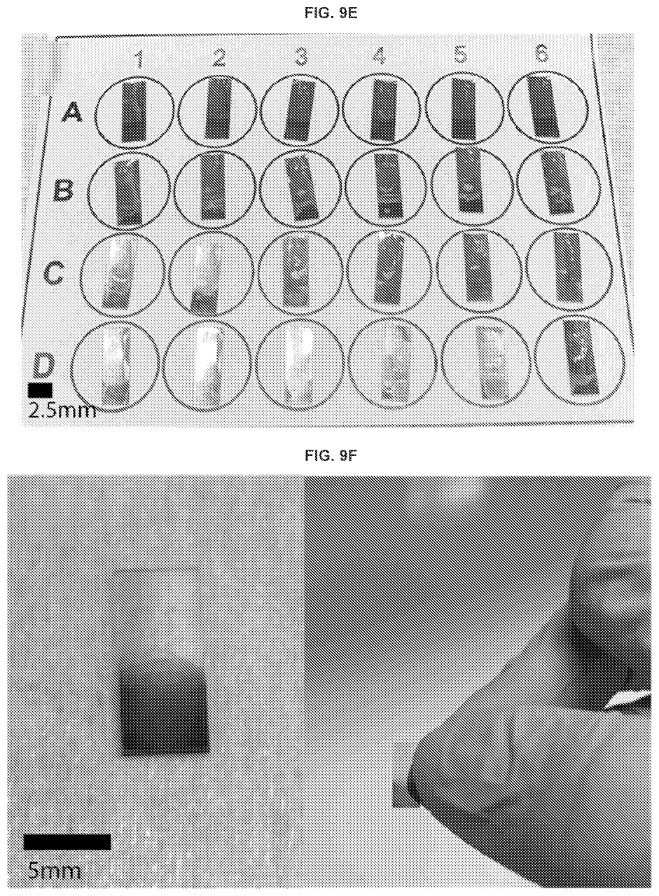

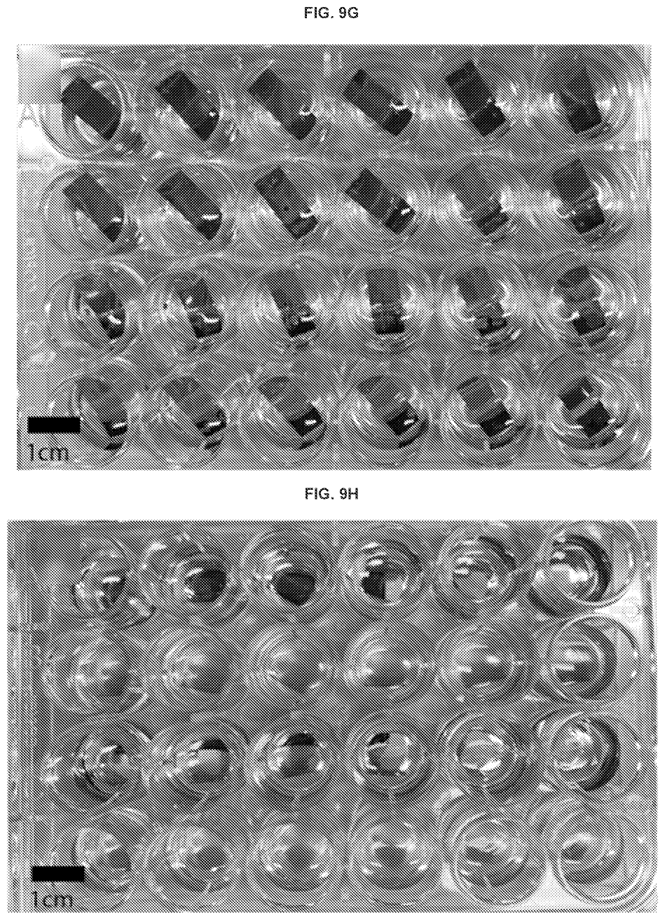

With the number of parameters, optimization was expedited by using gold strips as a model for electrodes. Gold strips were fabricated by coating polyimide tape in a metal evaporator, with 10 nm chromium (adhesion layer) and 250 nm gold. With regard to quantification, visual indication of polymerization (blackening of gold) provides immediate, albeit qualitative feedback (FIG. 8), helpful for quickly identifying failures. Electrical impedance is a second, quantitative, measure of success, which was performed. However, neither color nor impedance guarantees incorporation of HTL, particularly with EDOT/EDOT^{HTL} mixtures. Thus, an assay based on a custom recombinant protein was devised, in which HTP was fused to β-Lactamase (βLac). βLac is an enzyme that can cleave the indicator dye, CCF2, converting it from green to blue (Banala et al., 2013, ACS Chem. Biol., 8(10), 2127-2132). The enzymatic nature of the assay provides amplification (one βLac converts many CCF2 molecules), and the rate of dye conversion can be calibrated, providing estimates of the number of βLac enzymes over a wide dynamic range. The assay involves incubating PEDOT^{HTL} gold strips in purified HTP-βLac protein (HTP^{βLac}), followed by extensive washing. Each sample was submerged into 1 mL of CCF2 solution in a multi-well plate, and the green and blue intensities of the solution were measured via time-lapse fluorescence imaging (FIG. 9B). It was confirmed that no spontaneous conversion of CCF2 occurs over 24 hr; thus conversion can be attributed completely to HTP^{βLac}. To control for nonspecific protein adhesion, side-by-side assays were performed in which HTP^{βLac} was pre-blocked with a dummy HTL to prevent covalent capture by PEDOT^{HTL} (FIG. 9C).

The rate of CCF2 conversion produced was determined by known quantities of HTP-βLac, and it was confirmed that conversion by the βLac portion of the protein is unaffected by pre-blocking its HTP. This is important for two reasons. First, the faster rate of CCF2 conversion in positive (FIG. 9B) vs negative controls (FIG. 9C) provides proof that polymerized PEDOT^{HTL} can covalently capture HTP protein. Second, the quantitative nature of the assay has provided a robust and scalable platform which was used to improve the HTP binding capacity of PEDOT^{HTL} variants. A surface binding capacity of ˜10-fold higher than the HTP density on the surface of a neuron was targeted. From previous work with DART, it was estimated that neurons express ˜100 HTP proteins per μm² (Shields et al., 2017, Science, 356(6333)), and thus the goal was to capture ˜1,000 HTP-βLac molecules per μm². Scaling to the electro-polymerized area of our gold tape (2.5 mm×2.5 mm) yields ˜6 billion HTP-βLac molecules. For assay calibration, this number of purified HTP-βLac molecules was added to 1 mL of CCF2 solution (=10 pM HTP-βLac), and confirmed that conversion rate is linearly proportional to HTP-βLac concentration. Accordingly, it was calculated that an early design “condition 1.0” affords a density of ˜25 HTP-βLac per μm² (FIG. 9B, Condition 1.0). Since then, condition 2.0 was developed, with ˜100 HTP-βLac per μm² (FIG. 96, Condition 2.0). The additional binding afforded by condition 2.0 was due to an ethanol wash at the end of the polymerization procedure—this is the only difference between conditions 1.0 and 2.0. The ethanol wash removes any free EDOT or EDOT^{PEG3-HTL} monomers, allowing for more space between binding ligands (HTL), maximizing ability for HTL to HTP binding in the assay. PEDOT^{HTL} is a modular technology that builds upon the widely used PEDOT polymer, enabling compatibility with virtually all existing and future microelectrode devices.

Briefly the polymerization reactions are described by the following:

### EDOT Polymerization Methods

In order to make the EDOT^{-PEG[m]-HTL}:LiClO₄ polymerization solution, a 100 mM LiClO₄ Stock was made. All steps preformed under argon gas. The stock is made with 40 mL DI H₂O which has been argon bubbled in a 50 mL falcon tube. To the falcon tube, 425.6 mg LiClO₄ is added. The solution is then mixed at 1000 RPM for ˜15 minutes in an Eppendorf vortex mixer. This solution can range from 1 mM to 1M LiClO₄ stock and mixing times can vary to allow time for the LiClO₄ to get into solution. 100 mM LiClO₄ is an example. 3,333 mL of the LiClO₄ stock solution is added to a 10 μM EDOT^{-PEG[m]-HTL}aliquot in a 5 mL glass vial to make a 3.333 mM EDOT^{-PEG[m]-HTL}: 100 mM LiClO₄ solution. This solution is capped under argon and mixed using a stirbar in the vial overnight at 1150 rpm. 3.333 mM EDOT^{-PEG[m]-HTL} is an example. Solution molarity can range from 1 μM to 1M EDOT^{-PEG[m]-HTL}concentrations. Mixing times can vary from a few minutes to days depending on solubility. Henceforth this solution will be referred to as EDOT^{-PEG[m]HTL}:LiClO₄.

In order to make the EDOT^{-PEG[m]-HTL}:LiBF₄ polymerization solution, we first make a 100 mM LiBF₄ Stock. All steps preformed under argon gas. The stock is made with 40 mL DI H₂O which has been argon bubbled in a 50 mL falcon tube. To the falcon tube, 468.73 mg LiBF₄ is added. The solution is then mixed at 1000 RPM for ˜15 minutes in an Eppendorf vortex mixer. This solution can range from 1 mM to 1M L LiBF₄ stock and mixing times can vary to allow time for the LiBF₄ to get into solution. 100 mM LiBF₄ is an example. 3.333 mL of the LiBF₄stock solution is added to a 10 μM EDOT^{-PEG[m]-HTL} aliquot in a 5 mL glass vial to make a 3.333 mM EDOT^{-PEG[m]-HTL}: 100 mM LiBF₄ solution. This solution is capped under argon and mixed using a stirbar in the vial overnight at 1150 rpm. 3.333 mM EDOT^{-PEG[m]-HTL} is an example. Solution molarity can range from 1 μM to 1M EDOT^{-PEG[m]-HTL} concentrations. Mixing times can vary from a few minutes to days depending on solubility. Henceforth this solution will be referred to as EDOT^{-PEG[m]-HTL}:LiBF₄.

In order to make the EDOT^{-PEG[m]-HTL}:PSS polymerization solution, 20 mL DI H₂O is argon bubbled in a 25 mL glass vial. 129.6 μL PSS [Poly(sodium 4-styrenesulfonate) solution-here using average Mw ˜70,000, 30 wt. % in H₂O] to the vial under argon gas. This solution can range in stock concentration and mixing times can vary to allow time for the PSS to get into solution, 3.333 mL of the PSS stock solution is added to a 10 μM EDOT^{-PEG[m]-HTL} aliquot in a 5 mL glass vial to make a 3.333 mM EDOT^{-PEG[m]HTL}:PSS solution. 3.333 mM EDOT^{-PEG[m]-HTL} is an example. Solution molarity can range from 1 μM to 1M EDOT^{-PEG[m]-HTL} concentrations. Mixing times can vary from a few minutes to days depending on solubility. Henceforth this solution will be referred to as EDOT^{-PEG[m]-HTL}:PSS.

In order to make the EDOT:PSS polymerization solution, 20 mL DI H₂O is argon bubbled in a 25 mL glass vial. 129.6 μL PSS [Poly(sodium 4-styrenesulfonate) solution—here using average Mw ˜70,000, 30 wt. % in H₂O] to the vial under argon gas. Add 6.606 μL EDOT (3,4-Ethylenedioxythiophene, MW=142.18, 97%) to vial under argon gas to make a 10 mM EDOT solution. This solution can range from 1 nM to 1M EDOT solution and mixing times can vary to allow time for the EDOT to get into solution. Add stir bar to vial, cap under argon, and mix on stirplate β950 RMP overnight. Mixture method and time can vary depending on how quickly components go into solution. Specifics given here are merely an example. Henceforth this solution will be referred to as EDOT:PSS.

### Polymerization Cases

In the one-step polymerization cases, EDOT^{-PEG[m]-HTL}:LiClO₄ or EDOT^{-PEG[m]HTL} LiBF₄ or EDOT^{-PEG[m]-HTL}:PSS were polymerized at room temperature under argon (a no oxygen environment) at +1V vs. AgCl reference electrode for between 5 and 15 minutes. These polymerizations have been tested in standard room temperature and atmospheric conditions (i.e. without argon). These polymerizations can also be done at other voltage conditions between 0.5V to 2.5V for fixed voltage depositions and can also be done using cyclic voltammetry sweeps with ranges between −2.5V to 2.5V. In all cases, polymerization duration and voltages can extend beyond the example ranges given.

In these cases, EDOT^{-PEG[m]-HTL}:LiClO₄ or EDOT^{-PEG[m]-HTL}:LiBF₄ were the most successful although the polymers were thin and somewhat fragile during handling.

In the co-polymerization cases, EDOT^{-PEG[m]-HTL}:LiClO₁ or EDOT^{-PEG[m]-HTL}:LiBF₄ or EDOT^{-PEG[m]-HTL}:PSS were mixed with EDOT:PSS at varying percentages (0% to 100%) and polymerized at room temperature under argon (a no oxygen environment) at +1V vs. AgCl reference electrode for between 5 and 15 minutes. These polymerizations can also be done at other voltage conditions between 0.5V to 2.5V for fixed voltage depositions and can also be done using cyclic voltammetry sweeps with ranges between −2.5V to 2.5V. EDOT:PSS may be substituted for EDOT:LiClO₄ or EDOT:LiBF₄. In all cases, polymerization duration and voltages can extend beyond the example ranges given.

In some of these cases, there were clear instances of polymerization—as indicated by the visual darkening of the conductive material surface, characteristic of electropolymerization. In all cases, evaluating ligand incorporation was difficult to measure.

In the two-step polymerization cases, EDOT:PSS was polymerized onto the conductive material surface at +1V vs AgCl reference electrode for between 5 to 15 minutes under argon (a no oxygen environment). These polymerizations have been tested in standard room temperature and atmospheric conditions (i.e. without argon). These polymerizations can also be done at other voltage conditions between 0.5V to 2.5V for fixed voltage depositions and tested using cyclic voltammetry sweeps with ranges between −2.5V to 2.5V. After EDOT:PSS polymerization, EDOT^{-PEG[m]-HTL}:LiClO₄ or EDOT^{-PEG[m]-HTL}:LiBF₄ or EDOT^{-PEG[m]-HTL}:PSS were polymerized at room temperature under argon (a no oxygen environment) at +1V vs. AgCl reference electrode for between 5 and 15 minutes. These polymerizations have been tested in standard room temperature and atmospheric conditions (i.e. without argon). These polymerizations can also be done at other voltage conditions between 0.5V to 2.5V for fixed voltage depositions and can also be done using cyclic voltammetry sweeps with ranges between −2.5V to 2.5V. In all cases, polymerization duration and voltages can extend beyond the example ranges given.

Following polymerization, an ethanol wash of the polymer coated conductive material has been found to increase ligand availability for binding. This “post polymerization thinning” has been found to increase Halotag Protein binding to the polymer coated conductive material.

### Example 3

### Development of Engineered Conductive Polymer for Covalent Attachment to Neurons

The PEDOT-HTL interface to live neurons in culture will be characterized. EDOT-HTL conjugates have not been reported previously. While prior modifications of EDOT have been reported, this has often precluded electro-polymerization due to loss of monomer solubility and sidechain steric interference. To establish feasibility, candidate EDOT-HTL monomers have been synthesized, and validated that at least one can be electro-polymerized into PEDOT-HTL (FIG. 8).

With this starting point, the focus is to optimize PEDOT-HTL for genetically encoded electrophysiology, with the goal of maximizing HTP covalent binding capacity. The following will be explored: (i) Electrical parameters: current- vs voltage-clamp; cyclic vs constant; amplitude; duration; (ii) Chemical environment: solvent; pH; salts; counter ions; monomer concentrations; (iii) EDOT-HTL monomer design: HTL attachments site; linker length; linker composition; and (iv) EDOT/EDOT-HTL mixtures: various ratios and concentrations.

Many of the above parameters have precedent in the EDOT-conjugate literature. Electrical parameters and chemical environment are known to impact the organization of PEDOT polymers. Similarly, with regard to EDOT/EDOT-HTL mixtures, the reduction in steric hindrance provided by smaller EDOT monomers may promote longer polymer chains, thereby increasing total incorporation of EDOT-HTL moieties. Given the number of parameters, optimization has been expedited by using gold strips as a model for MEAs (multielectrode arrays). Gold strips were fabricated by coating polyimide tape in a metal evaporator, with 10 nm chromium (adhesion layer) and 250 nm gold, mirroring fabrication of MEAs. A cutting apparatus was devised to provide reproducible surface area, generating 462 samples per batch. With regard to quantification, visual indication of polymerization (blackening of gold) provides immediate, albeit qualitative feedback (FIG. 8), helpful for quickly identifying failures. Electrical impedance is a second, quantitative, measure of success that was performed. However, neither color nor impedance guarantees incorporation of HTL, particularly with EDOT/EDOT-HTL mixtures. Thus, an assay based on a custom recombinant protein was devised in which HTP is fused to β-Lactamase (βLac). βLac is an enzyme that can cleave the indicator dye, CCF2, converting it from green to blue. The enzymatic nature of the assay provides amplification (one βLac converts many CCF2 molecules), and the rate of dye conversion can be calibrated, providing estimates of the number of βLac enzymes over a wide dynamic range. The assay involves incubating PEDOT-HTL gold strips in purified HTP-βLac protein, followed by extensive washing. Each sample is then submerged into 1 mL of CCF2 solution in a multi-well plate, and the green and blue intensities of the solution measured via time-lapse fluorescence imaging (FIG. 96). It has been confirmed that no spontaneous conversion of CCF2 occurs over 24 hr; thus conversion can be attributed completely to HTP-βLac. To control for nonspecific protein adhesion, side-by-side assays were performed in which HTP-βLac was pre-blocked with a dummy HTL to prevent covalent capture by PEDOT-HTL (FIG. 9C). Finally, the rate of CCF2 conversion produced was determined by known quantities of HTP-βLac, and it was confirmed that conversion by the βLac portion of the protein was unaffected by pre-blocking its HTP. The faster rate of CCF2 conversion in positive (FIG. 9B) vs negative controls (FIG. 9C) provided proof that polymerized PEDOT-HTL can covalently capture HTP protein. In addition, the quantitative nature of the assay provided a robust and scalable platform for optimization of the HTP binding capacity of PEDOT-HTL.

The goal is to achieve a surface binding capacity ˜10-fold higher than the HTP density on the surface of a neuron. From prior work with DART, it is estimated that neurons express ˜100 HTP proteins per μm², and thus the goal is to capture ˜1,000 HTP-βLac molecules per μm². Scaling to the electro-polymerized area of the gold tape (2.5 mm×2.5 mm) yields ˜6 billion HTP-βLac molecules. For assay calibration, this number of purified HTP-βLac molecules was added to 1 mL of CCF2 solution (=10 μM HTP-βLac), and it was confirmed that conversion rate is linearly proportional to HTP-βLac concentration. Accordingly, that condition affords a density of ˜25 HTP-βLac per μm² (FIG. 96, Condition 1.0). Another condition demonstrated ˜100 HTP-βLac per μm² (FIG. 96, Condition 2.0), a 4-fold improvement.

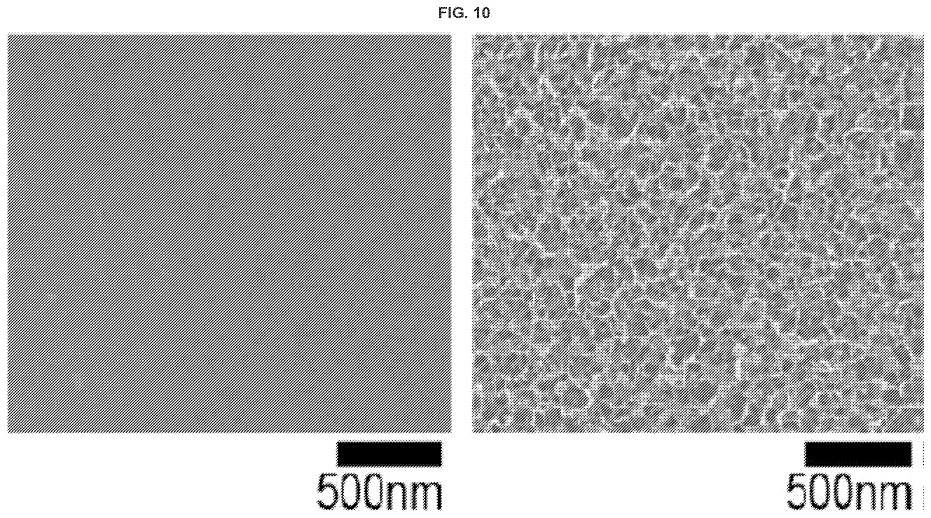

Although efforts have focused on smooth gold electrodes, there is interest in exploring electrode surfaces with complex topography and thus high surface area, with platinized-platinum being one of the most popular, yielding more than 200× increase in effective surface area (FIG. 10). Beyond optimization of HTL density, an alternative effort is to establish compatibility with other conductors. PEDOT has been successfully applied to tungsten, platinum, platinum iridium, tantalum pentoxide, titanium nitride (TiN), and the transparent conductor: indium tin oxide (ITO). Establishing compatibility with diverse substrates would maximize adoption and impact of the disclosure.

### Example 4

### Characterization of PEDOT-HTL Interface to Cultured Neurons

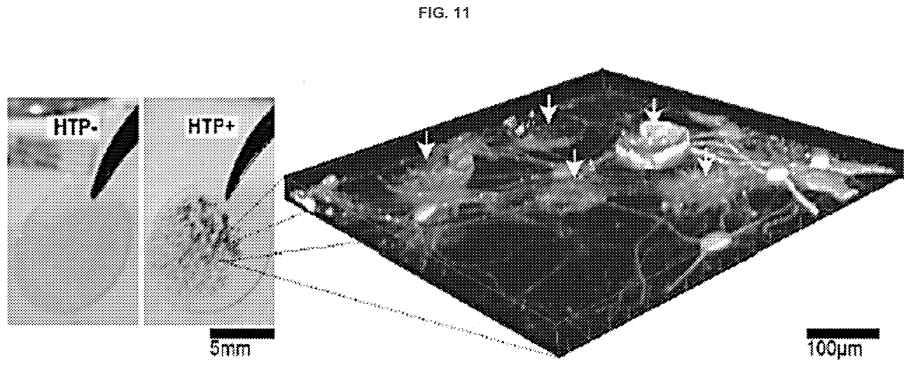

The data in FIG. 8 and FIGS. 9A-C establish that surface-immobilized PEDOT-HTL ligand can covalently capture freely diffusible HTP-βLac protein. Conversely, past work with DART establishes that if HTP protein is immobilized on a neuron, freely diffusible HTL ligand is likewise efficiently captured. Nevertheless, a previously untested scenario, in which neither HTL nor HTP is freely diffusible where HTP on a live neuron binds HTL on a hard surface, was tested. Commercially available HTL-beads (˜100 μm diameter; Promega) were sprinkled onto mature cultured neurons (14 days in vitro), with one coverslip containing HTP-positive (HTP+) neurons, and a second coverslip containing HTP-negative (HTP−) neurons. One day later, the coverslips were rinsed to remove unattached beads. A striking difference was seen: all HTL beads washed away on the HTP− coverslip, whereas immobilized HTL beads were so abundant on the HTP+ coverslip as to be visually apparent by the naked eye (FIG. 11, left side). The HTP+ coverslip was next imaged with a confocal microscope, noting that neurons expressing HTP on their surface also expressed a cytosolic fluorescent protein. Something quite surprising was observed: HTP+ neurons not only bound to the HTL beads, they physically changed their shape to wrap themselves around the beads (FIG. 11, right side). This data indicates that HTP on the surface of a neuron can bind to HTL on the hard surface of a 100 μm bead. Moreover, this suggests that physical coupling of HTP+ neurons onto PEDOT-HTL electrodes, comparable in size and hardness, is feasible.

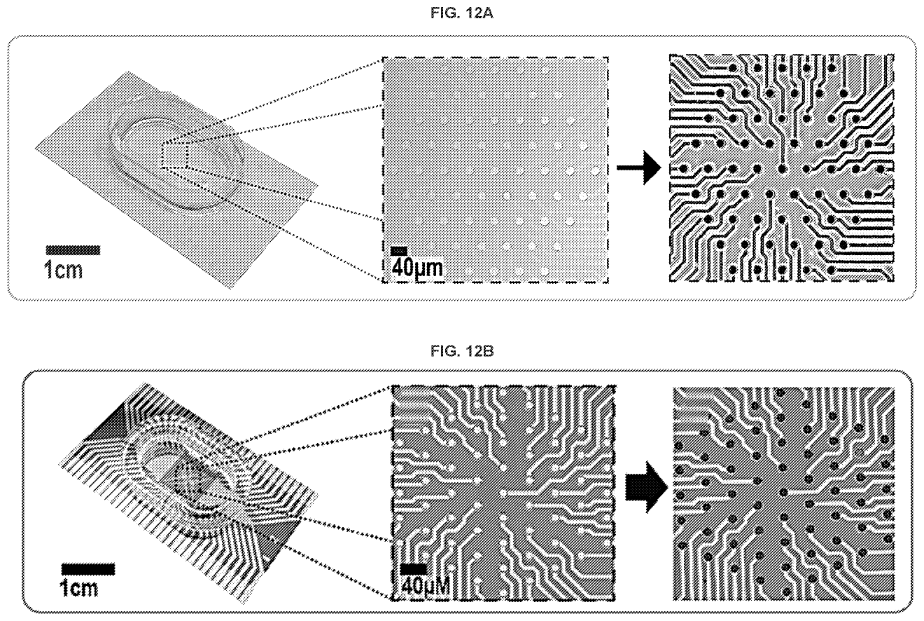

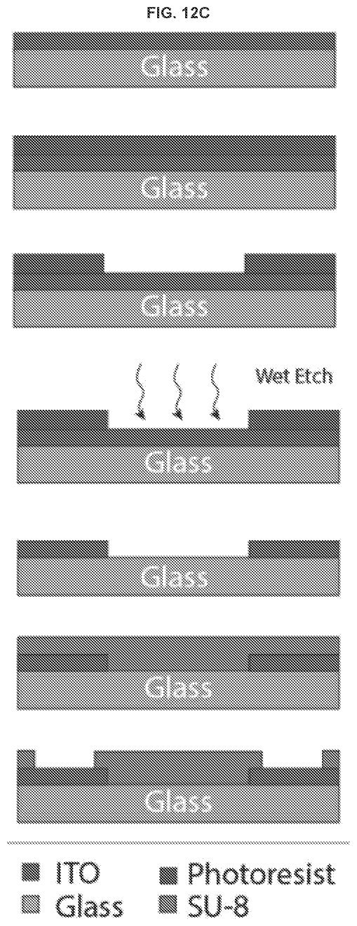

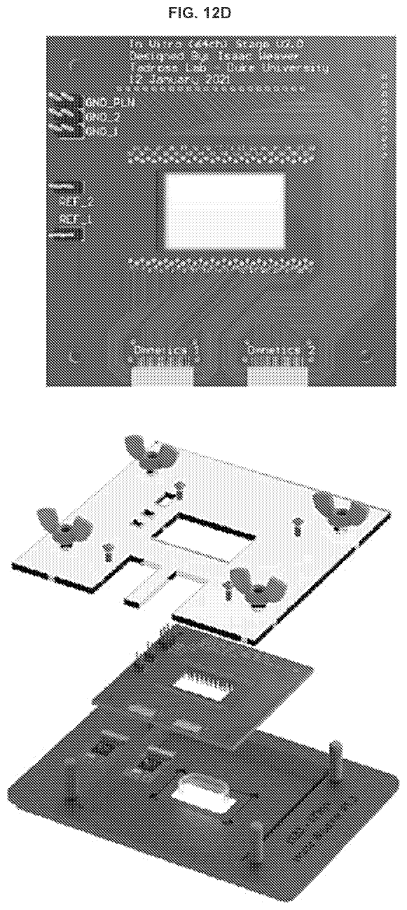

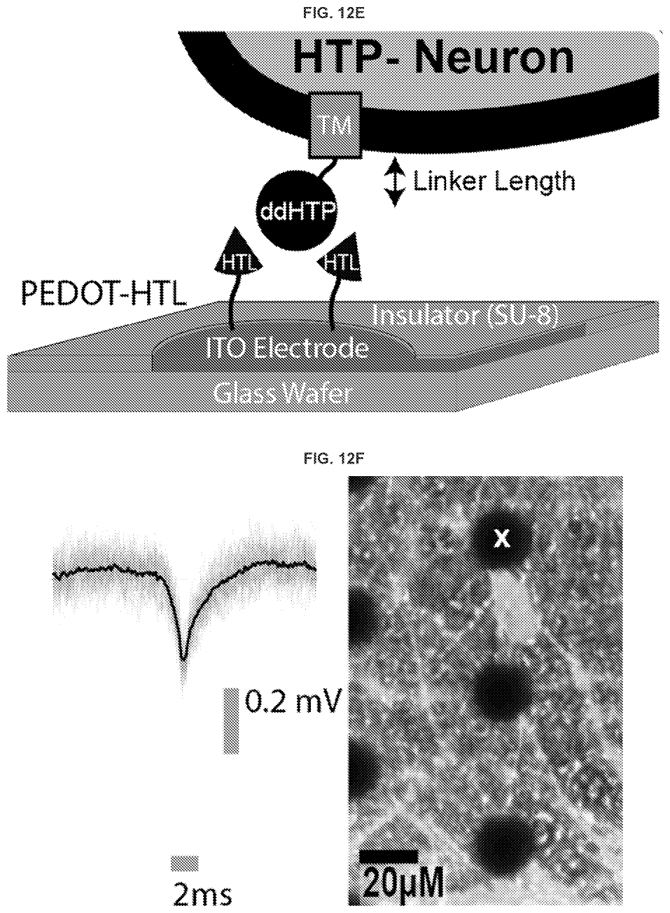

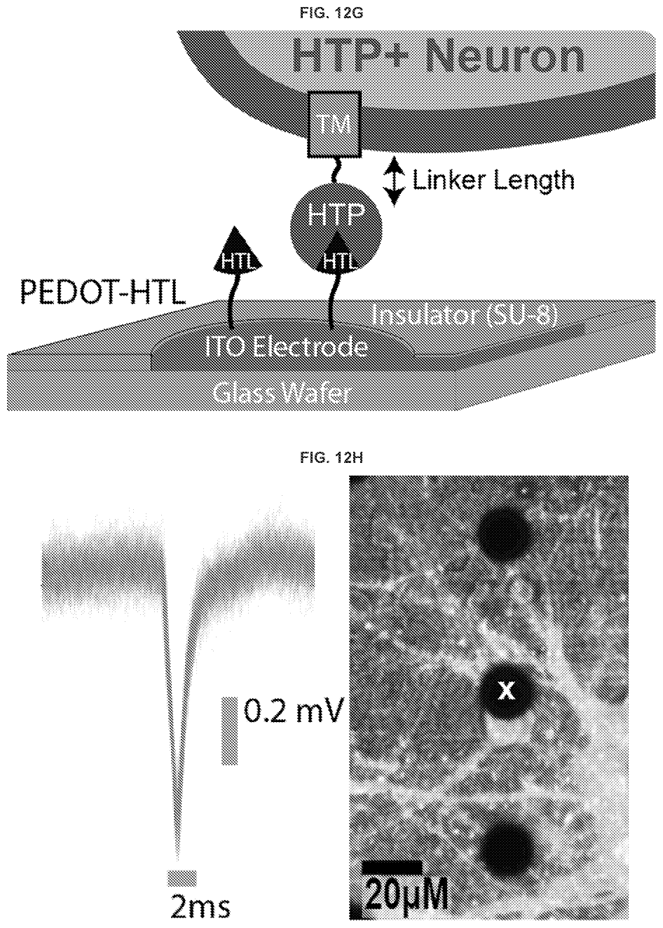

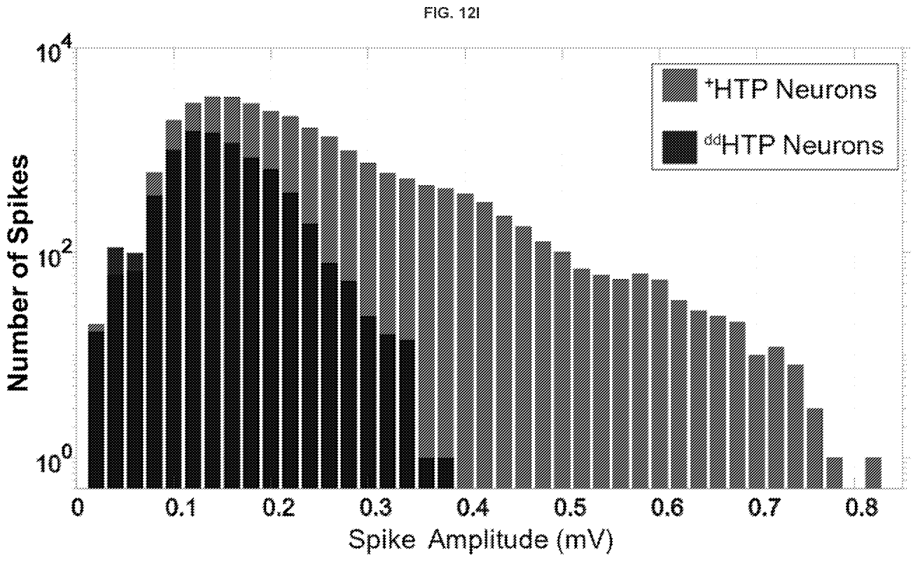

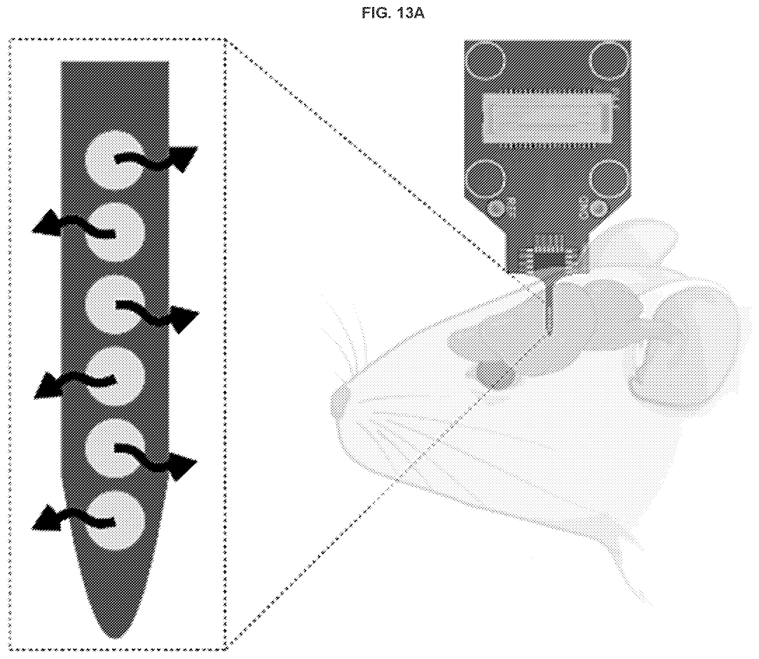

A typical neuron (˜10 μm soma diameter) is smaller than the pads of commercially available electrode arrays. Thus, a decision was made to design and fabricate MEAs, enabling customization of dimensions and materials. The original MEA devices consisted of silicon, silicon oxide (insulator), chromium (adhesion layer), gold (conductor), and SU-8 (insulator) (FIG. 12B). This design was replicated with transparent materials: borosilicate glass, indium tin oxide (ITO, conductor), and SU-8 (insulator) (FIG. 12A), enabling optical access on an inverted Olympus IX83 microscope. Polymerization of PEDOT-HTL on ITO proceeded equally well to gold substrate, validated with the HTP-βLac assay. One can fabricate ˜20 transparent MEAs per week, enabling a productive workflow for live-cell optimization.

A batch of freshly dissociated primary rat hippocampal neurons were divided into two groups, transfected with slightly different genetic constructs: HTP+ neurons are made to express an active surface HaloTag Protein, along with channelrhodopsin-2 (ChR2; opsin that drives action potentials in response to light), and GCaMP6s (green fluorescent calcium indicator). HTP− neurons were nearly identical, except for the use of a “double dead” HaloTag Protein (ddHTP), which is inactivated through targeted mutations to prevent binding to HTL. Cells from a given batch were seeded onto glass devices that had previously undergone identical PEDOT-HTL polymerization. Thus, HTP− (FIG. 12E) and HTP+(FIG. 12G) samples were identical except for the potential of covalent bond formation.

Following two weeks to allow neurons to mature, samples were mounted on an IX83 inverted microscope, connected to an Intan RHD 32-channel headstage and an OpenEphys acquisition system. Recordings were collected in the OpenEphys recording environment and analyzed offline using a custom Matlab script. A variety of stimulation conditions were explored and converged upon optogenetic illumination with low-intensity (0.7-4.5 mW) long-lasting (2 sec) blue light, combined with prevention of synaptic propagation with D-AP5, NBQX, and gabazine (to block excitatory and inhibitory synapses). This regime was favored because it produced sparse asynchronous activity, affording confidence that measured signals predominantly reflect action potentials from individual neurons.

The data show significant differences in HTP+ vs HTP− data obtained from 11 devices over two batches of neurons. In total, 36,105 spikes were detected, and plotted spike incidence as a function of magnitude (FIG. 12I). The most striking finding was the observation of large, 0.4-0.8 mV-amplitude signals from HTP+ samples. In contrast, the largest spike observed from HTP− samples was 0.37 mV, consistent with expectations for small-diameter MEA pads. In addition, it was noted a pronounced difference in the number of detected spikes: 8,040 spikes detected from 6 HTP− devices vs 28,065 spikes detected from 5 HTP+ devices, suggesting an approximately 3-fold improved ability to detect events above noise. It was confirmed that cell health was comparable on HTP- and HTP+ samples, with comparable proximity of neurons to MEA pads, and similar GCaMP responses (e.g., images in FIG. 12F vs FIG. 12H; green is ΔF of GCaMP signal).

While this data is promising, it is only the beginning of the design process. The parameters will be optimized to the unique neuronal context. The two parameters of interest include the length of the genetically encoded linker between HTP and the membrane (FIG. 12G), and the optimal electrode pad diameter. With regard to pad diameter, 5 μm, 10 μm, and 20 μm masks are available that are ready to be deployed with no process modification. With regard to linker length, a series of constructs have been cloned in which linker length is varied over an order of magnitude.

The goal is to achieve a bi-modal distribution in spike amplitude, such that electrical signals from covalently attached neurons can be unambiguously distinguished from non-attached cells. There was a hint of success for spike amplitudes larger than 0.4 mV, which are only observed on HTP+ samples. However, spikes in the 0.2-0.4 mV range cannot be unambiguously characterized as covalent, despite being more numerous on HTP+ samples (FIG. 12I). It is hypothesized that reducing pad diameter will reduce spike amplitude of HTP-samples, but that HTP+ amplitude may be relatively less impacted; thus smaller pads may accentuate the difference between HTP+vs HTP− samples. This may come at a cost, however, as smaller diameter pads may have a lower probability of conjugating to an HTP+ neuron. Similarly, there may be tradeoffs in optimal protein design: it is hypothesized that longer linkers will promote a higher probability of covalent attachment while shorter linkers may provide a more pronounced proximity-mediated amplitude enhancement.

Efforts have begun to explore high surface area metal coatings (platinized platinum and electrodeposited gold) in order to give the PEDOT-HTL a better anchor surface. Preliminary depositions indicate significant topography on the ITO electrodes (FIG. 10). Base polymer layers will be evaluated, including polyurethane to which the PEDOT-HTL can anchor, which has been shown to greatly improve mechanical stability with minimal effects on electrical conductivity.

### Example 5

### Characterization of PEDOT-HTL Interface to Neurons In Vivo

Finally, the technology will be applied to neurons in the CA1 hippocampal region of live mice. CA1 features densely packed neurons, maximizing probability of covalent attachment, and is central to questions spanning timescales of milliseconds (i.e., synaptic integration in memory) to months (progression of neurodegenerative disease). In particular, Alzheimer's Disease originates in the hippocampus, and it remains unknown how millisecond synaptic events evolve during months of disease progression. Such gaps in knowledge stem from technical challenges, which may be overcome with the genetically encoded electrophysiology described herein.

For in vivo characterization, the pyramidal cell layer of CA1 hippocampus, a nexus of conceptual interest in neuroscience, will be targeted. CA1 is home to the most densely packed neurons in the brain, maximizing the probability that an MEA would physically encounter a neuron. Coatings like Poly-D-Lysine are important for neuronal survival in 2D dissociated culture, but unnecessary in vivo owing to the rich supportive environment in a live 3D brain. While neurons in 2D culture survive at most for 4 weeks, neurons in vivo are generally healthier and can support stable AAV viral expression for months to years, enabling assessment of chronic stability. Moreover, gliosis can only be modeled in vivo. While gliosis typically interferes with recording stability, paradoxical improvement in the fidelity of electrical recordings have been observed when an electrode and neuron are encased by glial cells—a rare occurrence that may be promoted by the covalent attachment strategy disclosed herein.

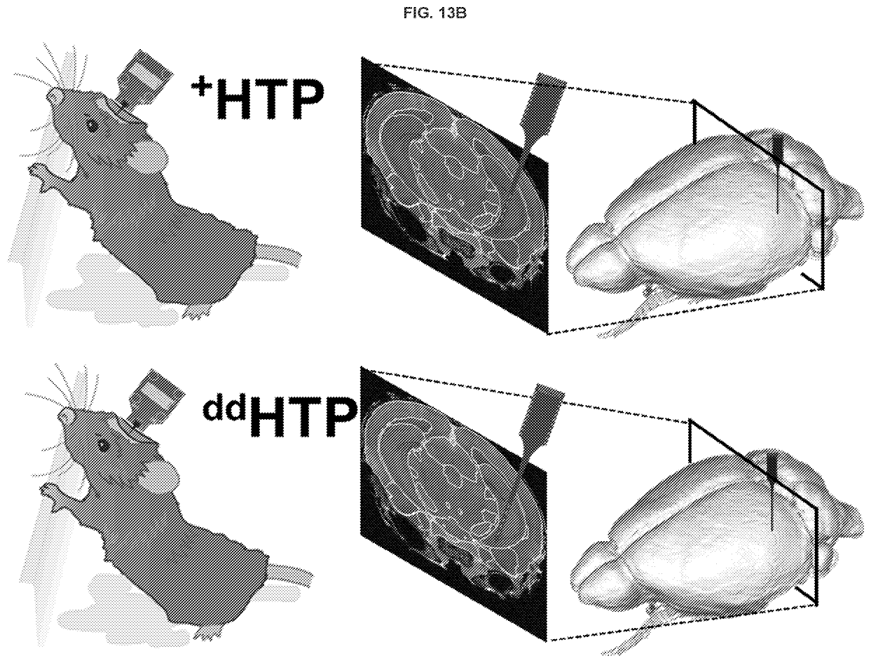

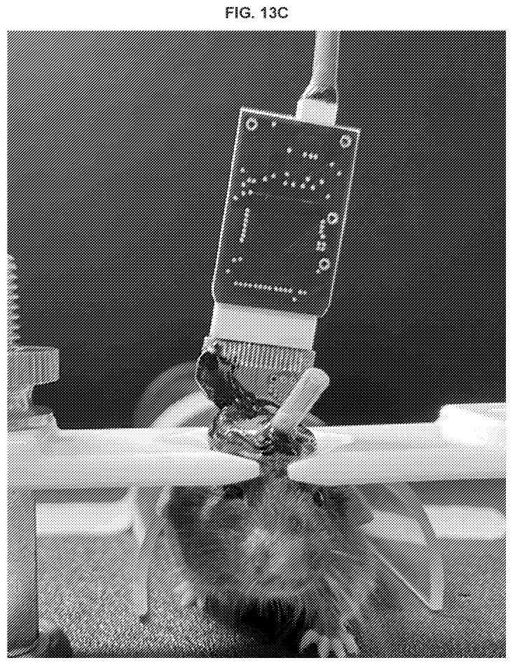

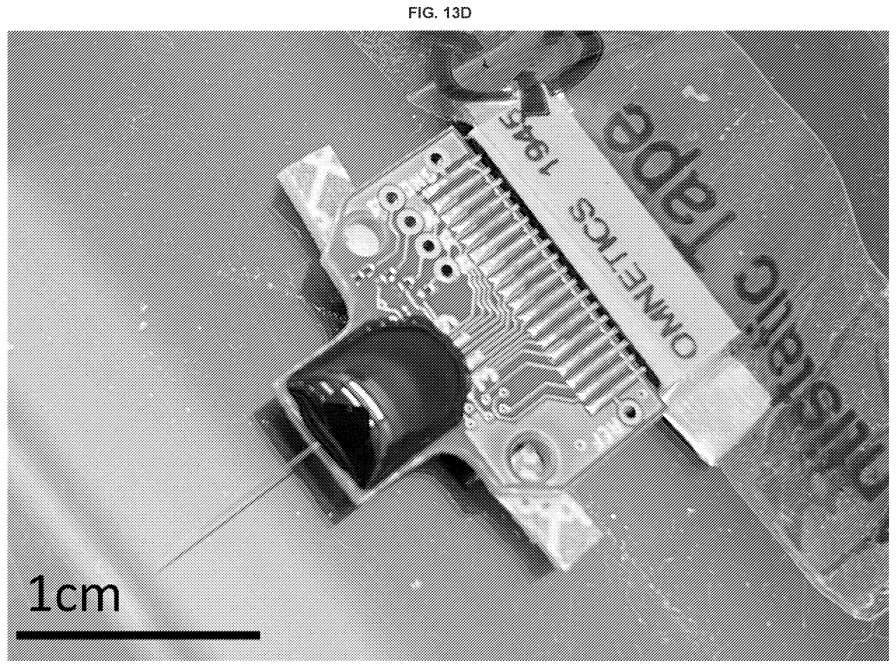

For these experiments, custom Michigan-style penetrating electrode arrays will be designed and fabricated. These arrays will be nearly identical to Example 4, differing mainly in shape and connector. Thus far, a 32-channel array has been designed, to be fabricated on a silicon wafer, etched into a probe shape, and packaged on a printed circuit board (PCB) headstage connector (FIGS. 13A-D). The PCB has been designed in Circuit Maker (Altium Ltd., San Diego, CA) and will be fabricated by a commercial foundry. The array will be attached to the PCB using gold wire bonding. After surface modification with PEDOT-HTL, the device will be sterilized with alcohol, similar to the procedure prior to culturing on MEAs disclosed in Example 4.

For a given device design, replicates will be made in batches of 24 to enable systematic characterization, Cohort design: CaMKIIα-Cre mice at postnatal day 21-24 (p21-24) will receive stereotaxic AAV viral injections targeting the CA1 region of the left dorsal hippocampus. Viral expression will be restricted to pyramidal neurons in CA1 owing to the expression of cre recombinase in the mouse strain. The validated cre-dependent HTP+ and HTP− AAV viruses, made for DART work, will be leveraged and repurposed herein (Shields et al., Science. 2017; 356(6333): eaaj2161. Each mouse (12 mice/virus with 6 mice/sex) will receive an HTP+ or control HTP− virus with the surgeon blinded to the viral condition. Arrays will be implanted with pads stereotaxically positioned to target the same CA1 region.

Following recovery from surgery, electrical recordings will be made using the Open-Ephys acquisition board (Open-Ephys, Lisbon, Portugal) wired to the Intan RHD 32-channel headstage (Intan Technologies, LLC, Culver City, CA), which clips to the implanted PCB connector. Recordings will be collected in the Open-Ephys recording environment and analyzed offline using a custom Matlab script, similar to that used in Example 4. Mice will be placed in a large open field arena and monitored with an overhead camera. Such environments are known to promote robust CA1 place-field activity. Recording will be repeated daily over the course of 2 months. Thereafter, mice will be deeply anesthetized with isofluorane and fixed via transcardial perfusion of PBS followed by 4% paraformaldehyde. Brains will be processed, cleared, stained with an anti-GFAP antibody (to label gliosis) and imaged to quantify whether each electrode pad is physically attached to an AAV-expressing neuron, and the extent and geometry of gliosis.

Traditional machine learning approaches will be used as well as modern deep learning methods to classify between HTP+ and HTP− mice based on features derived from electrophysiology data. Model efficacy will be evaluated by calculating classification accuracy and area under the receiver operating characteristic curve in a k-fold cross-validation scheme. It is hypothesized that electrical signals from covalently bound HTP+ mice will have a stronger intensity, higher SNR, and longer stability than HTP− mice. It is anticipated that electrical performance will correlate with physical neuron attachment to each PEDOT-HTL pad.

It is not necessary for every PEDOT-HTL pad to successfully couple to an HTP+ neuron, as one could restrict analysis to MEA pads with successful covalent attachment, inferred via histology. To improve the probability of successful coupling, common-sense solutions will be explored, including tuning the shape and area of MEA pads, and optimizing the surgical procedure to improve placement of AAV and MEA to the highest density location in CA1. Further examination as to whether pre-expression of AAV virus (˜2 weeks to allow robust HTP expression) prior to device insertion offers any benefit will be performed. Finally, neural growth factor coatings to promote neurite projections towards the electrode arrays will be evaluated. The decision to observe spontaneous CA1 activity was deliberate, as this offers a simple viral approach, where virtually every neuron expresses the same HTP+ or HTP− virus. If spontaneous activity is not sufficient to provide robust signals, then a combination of AAVs to drive expression of optogenetic or chemogenetic actuators may be used to enhance CA1 activity.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the disclosure are set out in the following numbered clauses:

Clause 1. A compound of formula (I)

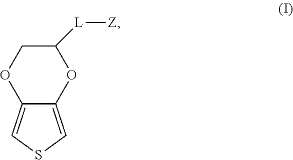

or a salt thereof, wherein: L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—; Z is a binding ligand selected from the group consisting of HaloTag, SNAP-tag, TMP-tag, βLac-tag, and CLIP-tag; and R^{a} is hydrogen or alkyl.

Clause 2. The compound of clause 1, wherein L is heteroalkylene-C(O)O— heteroalkylene-C(O)—, heteroalkylene-C(O)NR²-heteroalkylene-C(O)—, or heteroalkylene-OC(O)NR-heteroalkylene-C(O)—.

Clause 3. The compound of clause 1, wherein L is C₂₋₂₀heteroalkylene-C(O)O—C₂₋₆₀heteroalkylene-C(O)—, C₂₋₂₀heteroalkylene-C(O)NR^{a}—C₂₋₆₀heteroalkylene-C(O)—, or C₂₋₂₀heteroalkylene-OC(O)NR^{a}—C₂₋₆₀heteroalkylene-C(O)—.

Clause 4. The compound of clause 1, having formula (I-a)

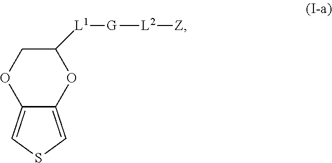

or a salt thereof, wherein: L¹ is C₂₋₁₀heteroalkylene; 0 is C(O)NR^{a} or C(O)O; L² is heteroalkylene-C(O)—; Z is a binding ligand selected from the group consisting of HaloTag, SNAP-tag, TMP-tag, βLac-tag, and CLIP-tag; and R^{a} is hydrogen or C₁₋₃alkyl.

Clause 5. The compound of clause 4, wherein: L¹ is C₂₋₆heteroalkylene; and L² is C₂₋₆₀ heteroalkylene-C(O)—.

Clause 6. The compound of clause 4, wherein L¹ is C₂₋₆heteroalkylene, wherein 1 carbon of the heteroalkylene is replaced by 0; and L² is C₂₋₆₀heteroalkylene-C(O)—, wherein at least 1 carbon of the heteroalkylene is replaced by O.

Clause 7. The compound of clause 1, having formula (I-b)

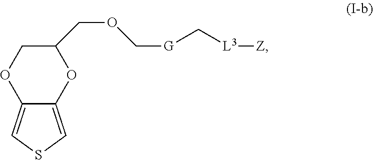

or a salt thereof, wherein: G is C(O)NR^{a} or C(O)O; L³ is (C—O—C)ₙ—C—C(O)—; Z is a binding ligand selected from the group consisting of HaloTag, SNAP-tag, TMP-tag, βLac-tag, and CLIP-tag; R^{a} is hydrogen or C₁₋₃alkyl; and n is 1 to 40.

Clause 8. The compound of clause 7, wherein: G is C(O)NH; L³ is (C—O—C)—C—C(O)—; Z is HaloTag, SNAP-tag, or CLIP-tag; and n is 1 to 10.

Clause 9. The compound of clause 1, having formula (I-c):

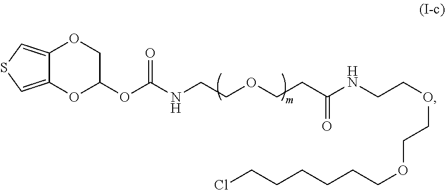

wherein: m is 1 to 40.

Clause 10. The compound of clause 1, selected from the group consisting of:

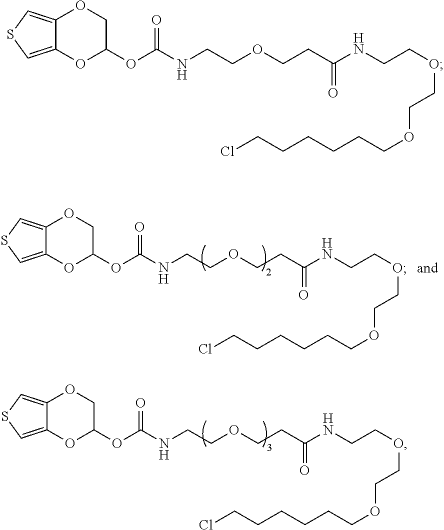

or a salt thereof.

Clause 11. A conductive polymer composition comprising a polymer and a dispersant, the polymer including recurring units of formula (II)

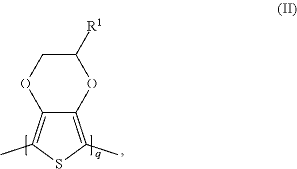

wherein: R¹ is hydrogen or

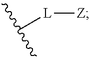

L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{u}, heteroalkylene-OC(O)NR^{a}, heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—; R is hydrogen or alkyl; Z is a binding ligand selected from the group consisting of HaloTag, SNAP-tag, TMP-tag, βLac-tag, and CLIP-tag; and q is 1 to 10,000,000.

Clause 12. The composition of clause 11, wherein the dispersant comprises polystyrene sulfonate (PSS), ClO₄⁻, BF₄⁻, PF₆⁻, bis(trifluoromethylsulfonyl)imide (BTFMSI), C₆H₅O₇⁻³, CO₃⁻², S₂O₃⁻², C₂H₃O₂⁻, HPO₄⁻², H₂PO₄⁻, Cl⁻, Br⁻, NO₃⁻, or a combination thereof.

Clause 13. The composition of clause 11 or clause 12, wherein the polymer is a copolymer comprising recurring unit of formula (II-a)

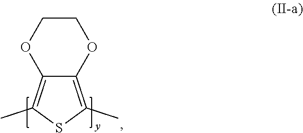

wherein y is 1 to 10,000,000; and recurring units of formula (II-b)

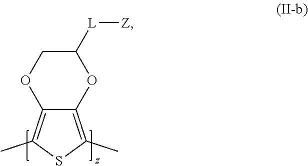

wherein z is 1 to 10,000,000.

Clause 14. The composition of any one of clauses 11-13, wherein: L is C₂₋₂₀heteroalkylene-C(O)O—C₂₋₆₀heteroalkylene-C(O)—, C₂₋₂₀heteroalkylene-C(O)NR^{a}—C₂₋₆₀heteroalkylene-C(O)—, or C₂₋₆₀heteroalkylene-OC(O)NR^{a}—C₂₋₆₀heteroalkylene-C(O)—; and Z is HaloTag, SNAP-tag, or CLIP-tag.

Clause 15. The composition of any one of clauses 11-14, wherein the composition includes the polymer and the dispersant at a concentration ratio of about 0.001:1 to about 10:1 (polymer:dispersant).

Clause 16. The composition of any one of clauses 11-15, wherein the dispersant comprises PSS, ClO₄⁻, BF₄⁻, or a combination thereof.

Clause 17. The composition of clause 13, wherein the copolymer is a random copolymer, an alternating copolymer, or a block copolymer.

Clause 18. A method of electrochemically synthesizing a conductive polymer, the method comprising adding a first mixture to an electrically conductive material, the first mixture comprising a solvent, a polystyrene sulfonate (PSS), and a 3,4-ethylenedioxythiophene (EDOT); applying an electrical current to the electrically conductive material to provide a poly(3,4-ethylenedioxythiophene) (PEDOT) on a surface of the electrically conductive material; adding a second mixture to the electrically conductive material, the second mixture comprising a solvent, a dispersant having a molecular weight of less than 1 kDa, and a compound of formula (I)

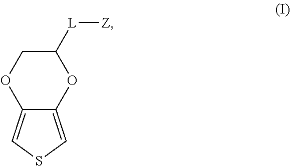

or a salt thereof, wherein: L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—; Z is a binding ligand selected from the group consisting of HaloTag, SNAP-tag, TMP-tag, βLac-tag, and CLIP-tag; and R^{a} is hydrogen or alkyl; and applying an electrical current to the electrically conductive material to provide a second polymer attached to the PEDOT, the second polymer including recurring units of formula (II-b)

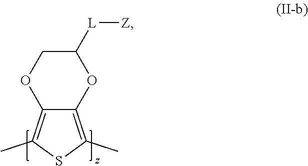

wherein: L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)F, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—; Z is a binding ligand selected from the group consisting of HaloTag, SNAP-tag, TMP-tag, βLac-tag, and CLIP-tag; R^{a} is hydrogen or alkyl; and z is 1 to 10,000,000.

Clause 19. The method of clause 18, wherein the dispersant of the second mixture comprises BF₄⁻, PF₆⁻, BTFMSI, C₆H₅O₇⁻³, CO₃⁻², S₂O₃⁻², C₂H₃O₂⁻, HPO₄⁻², H₂PO₄⁻, Cl⁻, Br⁻, NO₃⁻, or a combination thereof.

Clause 20. The method of clause 18 or clause 19, wherein: L is C₂₋₂₀heteroalkylene-C(O)O—C₂₋₆₀heteroalkylene-C(O)—, C₂₋₂₀heteroalkylene-C(O)NR^{a}—C₂₋₆₀heteroalkylene-C(O)—, or C₂₋₂₀heteroalkylene-OC(O)NR^{a}—C₂₋₂₀heteroalkylene-C(O)—; and Z is HaloTag, SNAP-tag, or CLIP-tag.

Clause 21. A microelectrode array comprising: an electrically conductive material; and a conductive polymer composition according to any one clauses 11-17 attached to a surface of the electrically conductive material.

Clause 22. The microelectrode array of clause 21, wherein the electrically conductive material is one or more of gold, platinized-platinum, tungsten, platinum, platinum iridium, tantalum pentoxide, titanium nitride, and indium tin oxide.

Clause 23. A method of detecting electrical activity in a cell, the method comprising: contacting a microelectrode array according to clause 21 with a cell; and detecting electrical activity in the cell by covalently attaching the polymer of the conductive polymer composition to a protein on the surface of the cell.

Clause 24. The method of clause 23, wherein the microelectrode array is implanted into a subject.

Clause 25. The method of clause 23 or clause 24, wherein the cell is capable of depolarizing.

Clause 26. The method of any one of clauses 23-25, wherein the cell is one or more of neurons, muscle cells, endocrine cells, keratinocytes, glia, and cell lines expressing voltage-gated ion channels.

Clause 27. The method of any one of clauses 23-26, wherein the protein on the surface of the cell is a genetically encoded protein.

Clause 28. The method of clause 27, wherein the genetically encoded protein is delivered to the cell with a virus.

Clause 29. The method of any one of clauses 23-28, wherein detecting the electrical activity comprises measuring a change in voltage, a change in current, or a combination thereof.

Clause 30. The method of clause 29, wherein the change in voltage, the change in current, or a combination thereof is used for decoding neural connectivity, frequency of firing, or a combination thereof.

Clause 31. The method of any one of clauses 23-30, wherein the protein on the surface of the cell is one or more of HaloTag protein, SNAP-tag protein, TMP-tag protein, βLac-tag protein, and CLIP-tag protein.

1. A compound of formula (I) 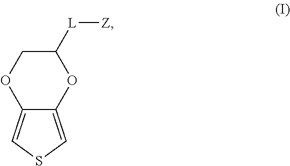
or a salt thereof, wherein:
L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-O(O)NR^{a}, heteroalkylene-OC(O)NR^{a}, heteroalkylene-C(O)O-heteroalkylene-C(O)—, heteroalkylene-C(O)NR³-heteroalkylene-C(O)—, or heteroalkylene-OC(O)NR¹-heteroalkylene-C(O)—;
Z is a binding ligand selected from the group consisting of HaloTag, SNAP-tag, TMP-tag, βLac-tag, and CLIP-tag; and
R^{a} is hydrogen or alkyl.

2. The compound of claim 1, wherein L is heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—.

3. The compound of claim 1, wherein L is C₂₋₂₀heteroalkylene-C(O)O—C₂₋₆₀heteroalkylene-C(O)—, C₂₋₂₀heteroalkylene-C(O)NR^{a}—C₂₋₆₀heteroalkylene-C(O)—, or C₂₋₂₀heteroalkylene-OC(O)NR^{a}—C₂₋₆₀heteroalkylene-C(O)—.

4. The compound of claim 1, having formula (I-a) 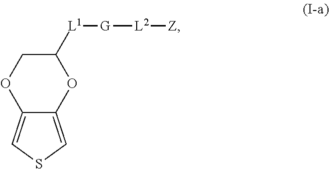
or a salt thereof, wherein:
L¹ is C₂₋₁₀heteroalkylene;
G is C(O)NR^{a} or C(O)O;
L² is heteroalkylene-C(O)—;
Z is a binding ligand selected from the group consisting of HaloTag, SNAP-tag, TMP-tag, βLac-tag, and CLIP-tag; and
R^{a} is hydrogen or C₁₋₃alkyl.

5. The compound of claim 4, wherein:
L¹ is C₂₋₆heteroalkylene; and
L² is C₂₋₆₀heteroalkylene-C(O)—.

6. The compound of claim 4, wherein
L¹ is C₂₋₅heteroalkylene, wherein 1 carbon of the heteroalkylene is replaced by O; and
L² is C₂₋₆₀heteroalkylene-C(O)—, wherein at least 1 carbon of the heteroalkylene is replaced by O.

7. The compound of claim 1, having formula (I-b) 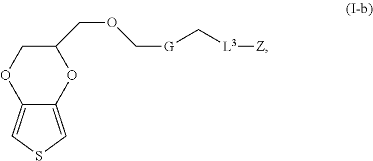
or a salt thereof, wherein:
G is C(O)NR^{a} or C(O)O;
L³ is (C—O—C)ₙ—C—C(O)—;
Z is a binding ligand selected from the group consisting of HaloTag, SNAP-tag, TMP-tag, βLac-tag, and CLIP-tag;
R^{a} is hydrogen or C₁₋₃alkyl; and
n is 1 to 40.

8. The compound of claim 7, wherein:
G is C(O)NH;
L³ is (C—O—C)ₙ—C—C(O)—;
Z is HaloTag, SNAP-tag, or CLIP-tag; and
n is 1 to 10.

9. The compound of claim 1, having formula (I-c): 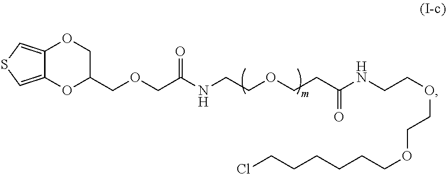
wherein:
m is 1 to 40.

10. The compound of claim 1, selected from the group consisting of: 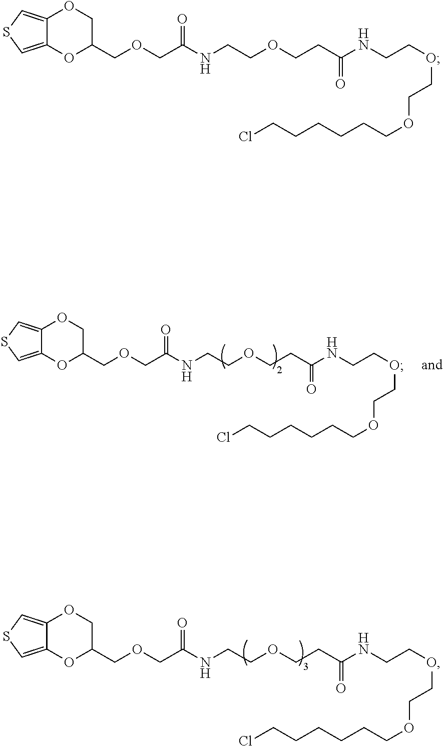
or a salt thereof.

11. A conductive polymer composition comprising a polymer and a dispersant, the polymer including recurring units of formula (II) 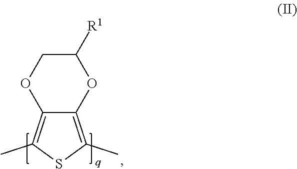
wherein:
R¹ is hydrogen or 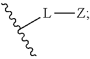
L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—;
R^{a} is hydrogen or alkyl;
Z is a binding ligand selected from the group consisting of HaloTag, SNAP-tag, TMP-tag, βLac-tag, and CLIP-tag; and
q is 1 to 10,000,000.

12. The composition of claim 11, wherein the dispersant comprises polystyrene sulfonate (PSS), ClO₄⁻, BF₄⁻, PF₆⁻, bis(trifluoromethylsulfonyl)imide (BTFMSI), C₆H₅O₇⁻³, CO₃⁻², S₂O₃⁻², C₂H₃O₂⁻, HPO₄⁻², H₂PO₄⁻, Cl⁻, Br⁻, NO₃⁻, or a combination thereof.

13. The composition of claim 11, wherein the polymer is a copolymer comprising recurring unit of formula (II-a) 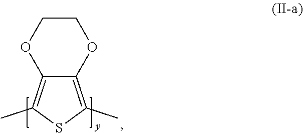
wherein y is 1 to 10,000,000; and
recurring units of formula (II-b) 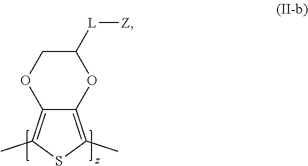
wherein z is 1 to 10,000,000.

14. The composition of claim 11, wherein:
L is C₂₋₂₀heteroalkylene-C(O)O—C₂₋₆₀heteroalkylene-C(O)—, C₂₋₂₀heteroalkylene-C(O)NR^{a}—C₂₋₆₀heteroalkylene-O(O)—, or C₂₋₂₀heteroalkylene-OC(O)NR^{a}—C₂₋₆₀heteroalkylene-C(O)—; and
Z is HaloTag, SNAP-tag, or CLIP-tag.

15. The composition of claim 11, wherein the composition includes the polymer and the dispersant at a concentration ratio of about 0.001:1 to about 10:1 (polymer:dispersant).

16. The composition of claim 11, wherein the dispersant comprises PSS, ClO₄⁻, BF₄⁻, or a combination thereof.

17. The composition of claim 13, wherein the copolymer is a random copolymer, an alternating copolymer, or a block copolymer.

18. A method of electrochemically synthesizing a conductive polymer, the method comprising
adding a first mixture to an electrically conductive material, the first mixture comprising a solvent, a polystyrene sulfonate (PSS), and a 3,4-ethylenedioxythiophene (EDOT);
applying an electrical current to the electrically conductive material to provide a poly(3,4-ethylenedioxythiophene) (PEDOT) on a surface of the electrically conductive material;
adding a second mixture to the electrically conductive material, the second mixture comprising a solvent, a dispersant having a molecular weight of less than 1 kDa, and a compound of formula (I) 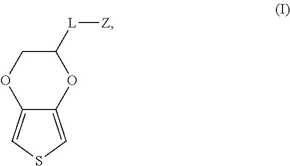
or a salt thereof, wherein:
L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—;
Z is a binding ligand selected from the group consisting of HaloTag, SNAP-tag, TMP-tag, βLac-tag, and CLIP-tag; and
R^{a} is hydrogen or alkyl; and
applying an electrical current to the electrically conductive material to provide a second polymer attached to the PEDOT, the second polymer including recurring units of formula (II-b) 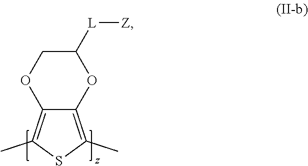
wherein:
L is alkylene, heteroalkylene, heteroalkylene-C(O)NR^{a}-heteroalkylene, heteroalkylene-C(O)O-heteroalkylene, heteroalkylene-OC(O)NR^{a}-heteroalkylene, alkylene-C(O)—, heteroalkylene-C(O)—, heteroalkylene-C(O)NR^{a}, heteroalkylene-OC(O)NR^{a}, heteroalkylene-C(O)O-heteroalkylene-C(O)—, heteroalkylene-C(O)NR^{a}-heteroalkylene-C(O)—, or heteroalkylene-OC(O)NR^{a}-heteroalkylene-C(O)—;
Z is a binding ligand selected from the group consisting of HaloTag, SNAP-tag, TMP-tag, βLac-tag, and CLIP-tag;
R^{a} is hydrogen or alkyl; and
z is 1 to 10,000,000.

19. The method of claim 18, wherein the dispersant of the second mixture comprises BF₄⁻, PF₅⁻, BTFMSI, C₅H₅O₇⁻³, CO₃⁻², S₂O₃⁻², C₂H₃O₂⁻, HPO₄⁻², H₂PO₄⁻, Cl⁻, Br⁻, NO₃⁻, or a combination thereof.

20. The method of claim 18, wherein:
L is C₂₋₂₀heteroalkylene-C(O)O—C₂₋₆₀heteroalkylene-C(O)—, C₂₋₂₀heteroalkylene-C(O)NR^{a}—C₂₋₆₀heteroalkylene-C(O)—, or C₂₋₂₀heteroalkylene-OC(O)NR^{a}—C₂₋₆₀heteroalkylene-C(O)—; and
Z is HaloTag, SNAP-tag, or CLIP-tag.

21. A microelectrode array comprising:
an electrically conductive material; and
a conductive polymer composition according to claim 11 attached to a surface of the electrically conductive material.

22. The microelectrode array of claim 21, wherein the electrically conductive material is one or more of gold, platinized-platinum, tungsten, platinum, platinum iridium, tantalum pentoxide, titanium nitride, and indium tin oxide.

23. A method of detecting electrical activity in a cell, the method comprising:
contacting a microelectrode array according to claim 21 with a cell; and
detecting electrical activity in the cell by covalently attaching the polymer of the conductive polymer composition to a protein on the surface of the cell.

24. The method of claim 23, wherein the microelectrode array is implanted into a subject.

25. The method of claim 23, wherein the cell is capable of depolarizing.

26. The method of claim 23, wherein the cell is one or more of neurons, muscle cells, endocrine cells, keratinocytes, glia, and cell lines expressing voltage-gated ion channels.

27. The method of claim 23, wherein the protein on the surface of the cell is a genetically encoded protein.

28. The method of claim 27, wherein the genetically encoded protein is delivered to the cell with a virus.

29. The method of claim 23, wherein detecting the electrical activity comprises measuring a change in voltage, a change in current, or a combination thereof.

30. The method of claim 29, wherein the change in voltage, the change in current, or a combination thereof is used for decoding neural connectivity, frequency of firing, or a combination thereof.

31. The method of claim 24, wherein the protein on the surface of the cell is one or more of HaloTag protein, SNAP-tag protein, TMP-tag protein, βLac-tag protein, and CLIP-tag protein.
